(19) United States
(12) Patent Application Publication
(10) Pub. No.: US 2026/0236741 A1
(43) Pub. Date: Aug. 13, 2026

(54) SYSTEMS AND METHODS FOR TRAINING A NEURAL FOUNDATION MODEL AND CONTROLLING A DEVICE USING A NEURAL FOUNDATION MODEL

(71) Applicant: Synchron Australia Pty Limited, Dover, DE (US)

(72) Inventors: Peter Eli YOO, Dover, DE (US); Nicholas F. HARDY, Dover, DE (US); Patrick Anthony HAJALI, Dover, DE (US); Thomas James OXLEY, Dover, DE (US)

(73) Assignee: Synchron Australia Pty Limited, Dover, DE (US)

(21) Appl. No.: 19/534,642

(22) Filed: Feb. 9, 2026

Related U.S. Application Data
(60) Provisional application No. 63/757,171, filed on Feb. 11, 2025.
(60) Provisional application No. 63/770,865, filed on Mar. 12, 2025.
(60) Provisional application No. 63/772,710, filed on Mar. 16, 2025.

(51) Int. Cl. G06N 3/045; G06F 3/01

(52) U.S. Cl. CPC G06N 3/045; G06F 3/015

(57) ABSTRACT

Systems and methods for training a machine learning model and controlling a device or software using the machine learning model are disclosed. For example, one such method comprises pre-training a machine learning model using unsupervised or self-supervised learning on sequences of neural signals recorded from electrodes. The method can also comprise post-training the pre-trained machine learning model using supervised learning on the sequences of neural signals or additional sequences of neural signals aligned with labeled contextual information to yield a post-trained machine learning model. The method can further comprise inputting real-time neural signals recorded from a user into the post-trained machine learning model to generate control information and controlling one or more devices based on the control information.

### CROSS-REFERENCE TO RELATED APPLICATIONS

This application claims the benefit of priority to U.S. Provisional Patent Application No. 63/757,171 filed on Feb. 11, 2025, U.S. Provisional Patent Application No. 63/770,865 filed on Mar. 12, 2025, and U.S. Provisional Patent Application No. 63/772,710 filed on Mar. 16, 2025, the contents of which are incorporated herein by reference in their entireties.

### TECHNICAL FIELD

This disclosure relates generally to the intersection of machine learning and brain-computer interfaces and, more specifically, to systems and methods for training a neural foundation model and controlling a device or a software application using the neural foundation model.

### BACKGROUND

Conventional brain-computer interface (BCI) systems suffer from fundamental technical limitations that prevent scalability across tasks, environments, and users. Existing BCI approaches typically rely on task-specific, supervised decoding pipelines that require predefined labels, hand-designed features, and per-user calibration, resulting in machine learning models that generalize poorly beyond the narrow conditions under which they were trained. Contextual information, when used at all, is treated as static metadata or is required continuously at inference, creating brittle systems that degrade when sensors, environments, or user behaviors change. These approaches also depend on labor-intensive data labeling and yield low signal separability when relying solely on passive neural observation, making it difficult to reliably capture diverse cognitive states at scale. As a result, current BCI systems do not support efficient population-level training, rapid onboarding of new users, or reuse of learned representations across devices or applications.

Moreover, most commercially-available machine learning models are trained only on textual data. This textual data, which is encoded in human language, is a lossy compressed form of human thought and cognition. Thus, most commercially-available machine learning models are not optimized for use with BCI systems.

Therefore, a new type of machine learning model is needed that can directly be trained on neural signals. Such a neural machine learning model should be capable of being further trained or post-trained on a combination of neural signals and contextual information. Such a neural machine learning model can then be integrated with a BCI system and used to perform helpful tasks such as controlling external devices or software applications running on such external devices. Such a neural machine learning model should also be able to overcome the challenges discussed above without binding the model to specific tasks, environments, or users, thereby enabling robust generalization and scalable deployment.

### SUMMARY

Disclosed herein are systems and methods for training a neural foundation model and controlling a device or a software application using the neural foundation model. In one aspect, a method of controlling one or more devices is disclosed. The method can comprise pre-training a machine learning model using unsupervised or self-supervised learning on sequences of neural signals prior to associating the sequences of neural signals with task-specific labels to yield a pre-trained machine learning model. The sequences of neural signals can be recorded from electrodes implanted within or positioned on one or more subjects. The machine learning model can learn task-agnostic latent representations of neural activity of the one or more subjects. The method can also comprise post-training the pre-trained machine learning model using supervised learning on the sequences of neural signals or additional sequences of neural signals aligned with labeled contextual information to yield a post-trained machine learning model. The labeled contextual information can be obtained by recording contextual data using one or more modalities in real or simulated environments experienced by the one or more subjects or one or more users. The labeled contextual information can include descriptive representations derived from the one or more modalities obtained by passively observing an environment or activities of the one or more subjects or the one or more users and/or modifying the environment of the one or more subjects or the one or more users to induce targeted neural activity. During the post-training, the pre-trained machine learning model can transform the task-agnostic latent representations learned during the pre-training into brain-state embeddings configured to align neural and contextual representations. The method can further comprise inputting real-time neural signals recorded from one of the users into the post-trained machine learning model to generate control information based on the brain-state embeddings and controlling one or more devices based on the control information.

In another aspect, a method of controlling one or more devices is disclosed. The method can comprise providing a pre-trained machine learning model and post-training the pre-trained machine learning model using supervised learning on sequences of neural signals aligned with labeled contextual information to yield a post-trained machine learning model. The labeled contextual information can be obtained by recording contextual data from one or more modalities in real or simulated environments experienced by one or more subjects or one or more users. The labeled contextual information can comprise descriptive representations derived from the one or more modalities obtained by passively observing an environment or activities of the one or more subjects or the one or more users and/or modifying the environment of the one or more subjects or the one or more users to induce targeted neural activity. During the post-training, the pre-trained machine learning model can transform task-agnostic latent representations learned during pre-training into brain-state embeddings configured to align neural and contextual representations. The method can also comprise inputting real-time neural signals recorded from one of the users into the post-trained machine learning model to generate control information based on the brain-state embeddings and controlling one or more devices based on the control information.

In a further aspect, a method of controlling one or more devices is disclosed. The method can comprise inputting real-time neural signals recorded from a user into a post-trained machine learning model to generate control information based on brain-state embeddings and controlling one or more devices based on the control information. The post-trained machine learning model can be obtained by pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model. The pre-trained machine learning model can then be post-trained using supervised learning on the sequences of neural signals or additional sequences of neural signals aligned with labeled contextual information to yield the post-trained machine learning model. The labeled contextual information can be obtained by recording contextual data from one or more modalities in real or simulated environments experienced by the one or more subjects or one or more users. The labeled contextual information can include descriptive representations derived from the one or more modalities obtained by passively observing an environment or activities of the one or more subjects or the one or more users and/or modifying the environment of the one or more subjects or the one or more users to induce targeted neural activity. During the post-training, the pre-trained machine learning model can transform the task-agnostic latent representations learned during the pre-training into the brain-state embeddings configured to align neural and contextual representations.

In an additional aspect, a system for controlling one or more devices is disclosed. The system can comprise a recording device configured to record real-time neural signals of a user and a control unit comprising one or more memory units including instructions stored thereon and one or more processors. The one or more processors can be communicatively coupled to the one or more memory units. The control unit can be configured to receive or obtain the real-time neural signals from the recording device. The one or more processors can be programmed to execute the instructions to input the real-time neural signals into a post-trained machine learning model to generate control information based on brain-state embeddings and control one or more devices based on the control information. The post-trained machine learning model can be obtained by pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model. The pre-trained machine learning model can then be post-trained using supervised learning on the sequences of neural signals or additional sequences of neural signals aligned with labeled contextual information to yield the post-trained machine learning model. The labeled contextual information can be obtained by recording contextual data from one or more modalities in real or simulated environments experienced by the one or more subjects or one or more users. The labeled contextual information can include descriptive representations derived from the one or more modalities obtained by passively observing an environment or activities of the one or more subjects or the one or more users and/or modifying the environment of the one or more subjects or the one or more users to induce targeted neural activity. During the post-training, the pre-trained machine learning model can transform the task-agnostic latent representations learned during the pre-training into the brain-state embeddings configured to align neural and contextual representations.

In yet another aspect, a non-transitory computer-readable medium is disclosed. The non-transitory computer-readable medium can include instructions stored thereon that, when executed by one or more processors, can cause the one or more processors to perform operations including inputting real-time neural signals recorded from a user into a post-trained machine learning model to generate control information based on brain-state embeddings and controlling one or more devices based on the control information. The post-trained machine learning model can be obtained by pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model. The pre-trained machine learning model can then be post-trained using supervised learning on the sequences of neural signals or additional sequences of neural signals aligned with labeled contextual information to yield the post-trained machine learning model. The labeled contextual information can be obtained by recording contextual data from one or more modalities in real or simulated environments experienced by the one or more subjects or one or more users. The labeled contextual information can include descriptive representations derived from the one or more modalities obtained by passively observing an environment or activities of the one or more subjects or the one or more users and/or modifying the environment of the one or more subjects or the one or more users to induce targeted neural activity. During the post-training, the pre-trained machine learning model can transform the task-agnostic latent representations learned during the pre-training into the brain-state embeddings configured to align neural and contextual representations.

The machine learning model can be a neural foundation model.

The task-agnostic latent representations can be internal representations learned by the machine learning model during the pre-training that encode structure, patterns, and relationships present in the sequences of neural signals without relying on the task-specific labels or predefined outputs. The task-agnostic latent representations can be distinct from raw neural signals, device control commands, or the task-specific labels. The sequences of neural signals can be recorded from the electrodes implanted within or positioned on the one or more subjects.

In some embodiments, pre-training the machine learning model can further comprise intentionally excluding task labels associated with the sequences of neural signals.

In some embodiments, pre-training the machine learning model can be performed without requiring the machine learning model to output a task identifier, a command label, or a device control action.

In some embodiments, pre-training the machine learning model can further comprise providing the sequences of neural signals recorded from the electrodes as inputs to the machine learning model and obtaining, as outputs, autoregressive predictions, masked neural signal predictions, or contrastive learning predictions of upcoming or ensuing neural signals.

In some embodiments, the sequences of neural signals provided as inputs to the machine learning model can be provided as discretized neural signals treated as tokenized inputs.

In some embodiments, passively observing the environment or the activities of the one or more subjects or the one or more users can further comprise observing the environment or the activities of the one or more subjects or the one or more users without intentionally modifying the environment of the one or more subjects or the one or more users or interfering with the activities of the one or more subjects or the one or more users. In these and other embodiments, passively observing the environment or the activities of the one or more subjects or the one or more users can also comprise recording the contextual data using the one or more modalities by recording data or information concerning a physical environment, a virtual environment, or an augmented environment experienced by the one or more subjects or the one or more users.

In some embodiments, modifying the environment of the one or more subjects or the one or more users can further comprise intentionally modifying a virtual environment or an augmented environment of the one or more subjects or the one or more users to induce the targeted neural activity and recording the contextual data using the one or more modalities further comprises recording modifications to the virtual environment or the augmented environment of the one or more subjects or the one or more users.

In some embodiments, intentionally modifying the environment of the one or more subjects or the one or more users can further comprise intentionally modifying the virtual environment of the one or more subjects or the one or more users via a virtual reality device worn by one of the subjects or one of the users or intentionally modifying the augmented environment of the one or more subjects or the one or more users via an augmented reality device worn by one of the subjects or one of the users.

In some embodiments, the contextual data can be recorded automatically using one or more sensors, system logs, or instrumentation without manual annotation.

In some embodiments, inputting the real-time neural signals into the post-trained machine learning model can be undertaken without any real-time contextual information being inputted into the post-trained machine learning model to generate the control information.

In other embodiments, inputting the real-time neural signals into the post-trained machine learning model can be undertaken with real-time contextual information also being inputted into the post-trained machine learning model to generate the control information.

In some embodiments, the post-trained machine learning model can further comprise one or more classifiers, one or more decoder heads, or a combination thereof.

The post-trained machine learning model can accomplish different tasks without having to re-train the machine learning model. Moreover, the post-trained machine learning model can be used by different users without having to re-train the machine learning model.

In some embodiments, controlling the one or more devices based on the control information can further comprise transmitting device control signals that cause the one or more devices to perform or cease from performing a physical or operational action.

In some embodiments, the brain-state embeddings can modulate, gate, delay, or parameterize the control information.

In some embodiments, the one or more devices can comprise at least one of a computing device, a robotic device, an assistive device, a smart-home device, an Internet-of-Things (IoT) device, and a mobility vehicle.

### BRIEF DESCRIPTION OF THE DRAWINGS

FIG. 1A illustrates a method of pre-training a machine learning model as part of an unsupervised or self-supervised training phase.

FIG. 1B illustrates a method of post-training the pre-trained machine learning model as part of a supervised training phase.

FIG. 1C illustrates a method of using the post-trained machine learning model to control one or more devices.

FIG. 2A illustrates one embodiment of a system that can be used to train a machine learning model.

FIG. 2B illustrates one embodiment of a recording device of the system implemented as a stent-electrode array comprising a plurality of electrodes.

FIG. 2C illustrates that a communication conduit can connect the stent-electrode array with a telemetry unit communicatively coupled to a computing device of the system.

FIG. 2D illustrates a close-up view of an embodiment of the telemetry unit.

FIG. 3A illustrates another embodiment of a recording device implemented as a coiled wire comprising a plurality of electrodes.

FIG. 3B illustrates yet another embodiment of a recording device implemented as an anchored wire comprising a plurality of electrodes.

FIG. 3C illustrates one embodiment of an electroencephalogram (EEG) device or EEG cap that can serve as the recording device.

FIG. 3D illustrates one embodiment of an electrocorticography (ECoG) device that can serve as the recording device.

FIG. 3E illustrates one embodiment of a functional magnetic resonance imaging (fMRI) device that can serve as the recording device.

FIG. 3F illustrates one embodiment of a functional near infrared spectroscopy (fNIRS) device that can serve as the recording device.

FIG. 4A illustrates one example implementation of a method of pre-training and post-training a machine learning model using neural signals discretized as neural tokens.

FIG. 4B illustrates true oscillatory burst patterns extracted from neural data as well as the corresponding predicted burst probabilities outputted by the pre-trained machine learning model.

FIG. 4C illustrates a validation loss curve produced during the pre-training phase of the neural foundation model.

FIG. 4D is a bar chart comparing the performance of multiple machine learning models as it pertains to the accuracy of a click task.

FIG. 5A illustrates one embodiment of a virtual-reality (VR) environment rendered via a VR headset.

FIG. 5B illustrates one embodiment of an augmented-reality (AR) environment as viewed through an AR headset.

FIG. 5C illustrates one embodiment of a graphical environmental as viewed on a screen display.

FIG. 6 illustrates a user controlling a smart device while wearing one embodiment of wearable display device to view the smart device.

FIG. 7A illustrates one embodiment of an action list user interface (UI) viewed through a wearable display device and comprising a plurality of possible actions that a user can select from the action list UI.

FIG. 7B illustrates the user selecting one of the plurality of possible actions from the action list UI.

FIG. 8A illustrates another embodiment of an action list UI viewed through a wearable display device and the user selecting one of the plurality of possible actions from the action list UI.

FIG. 8B illustrates yet another embodiment of an action list UI viewed through a wearable display device and the user selecting one of the plurality of possible actions from the action list UI.

FIG. 9 illustrates a plurality of brain states corresponding to reproducible neural activity that can be detected in different regions of the brain.

### DETAILED DESCRIPTION

Disclosed herein is a neural foundation model pre-trained on unlabeled neural signal sequences and subsequently post-trained using neural signals aligned with contextual information. The post-trained neural foundation model can enable inference of brain states or brain-state embeddings and control of devices using neural signals without contextual inputs at inference (or, optionally, with contextual inputs at inference). The post-trained neural foundation model can be deployed in practical environments to enable robust, low-latency, and reliable brain-driven control of external devices.

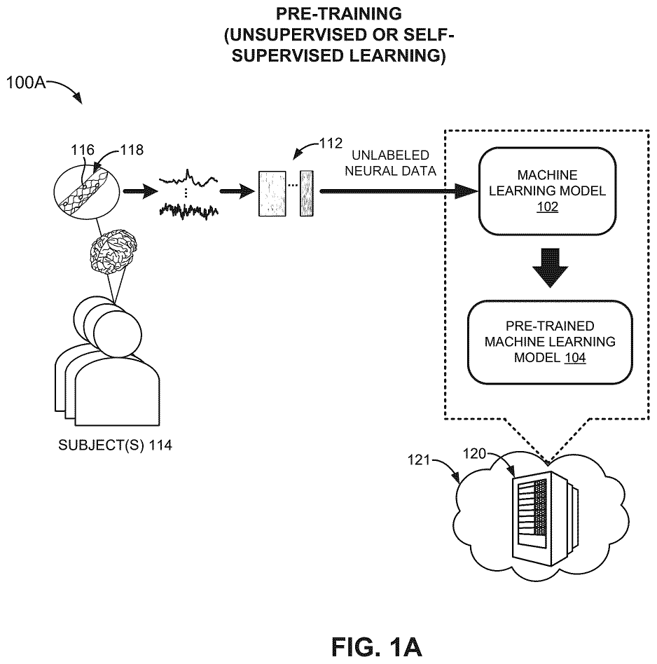

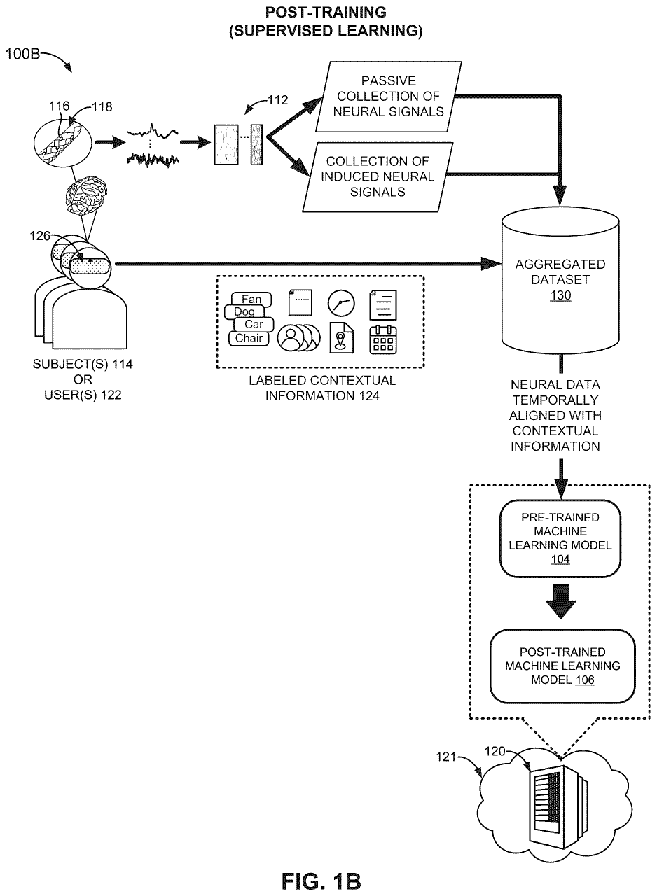

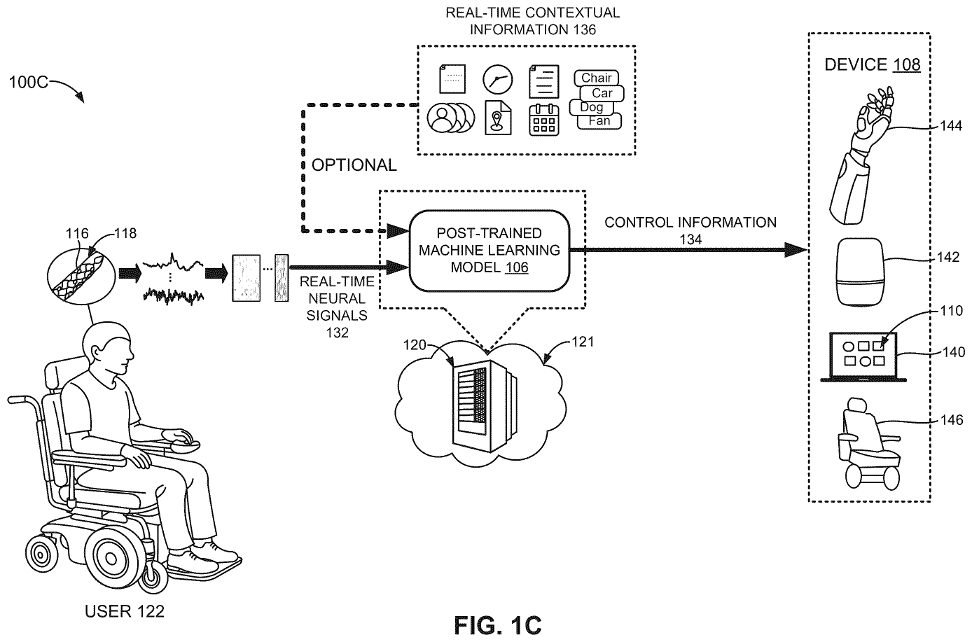

FIG. 1A-1C illustrate example methods of training (e.g., pre-training and post-training) the neural foundation model and using the neural foundation model to control one or more devices or software applications running on such devices. More specifically, FIG. 1A illustrates an example method 100A of pre-training a machine learning model 102 as part of an unsupervised or self-supervised training phase to yield a pre-trained machine learning model 104. FIG. 1B illustrates an example method 100B of post-training the pre-trained machine learning model 104 as part of a supervised training phase to yield a post-trained machine learning model 106. FIG. 1C illustrates an example method 100C of using the post-trained machine learning model 106 to control one or more devices 108 or software applications 110 running on such devices 108.

In some embodiments, methods 100A, 100B, and 100C can be considered part of one overall method. In other embodiments, the methods 100A and 100B can be considered part of a method of training a machine learning model and method 100C can be considered a method of deploying the machine learning model. In further embodiments, methods 100B and 100C can be considered part of a method of post-training a previously pre-trained machine learning model and subsequently deploying the post-trained machine learning model. In these embodiments, a pre-trained machine learning model is provided and method 100B comprises post-training the provided pre-trained machine learning model 104.

FIG. 1A illustrates that a machine learning model 102 can first be pre-trained on sequences of neural signals 112 recorded from one or more subjects 114 prior to associating the sequences of neural signals 112 with task-specific labels such that the machine learning model 102 learns task-agnostic latent representations of neural activity to yield a pre-trained machine learning model 104. The sequences of neural signals 112 can be provided as inputs to the machine learning model 102 without any task-specific labels. Since the machine learning model 102 is first pre-trained on sequences of neural signals 112 without task-specific labels, the machine learning model 102 can be considered a neural foundation model.

In some embodiments, the machine learning model 102 can be a transformer-based model or have a transformer architecture configured to process sequences of neural signals using attention mechanisms. In other embodiments, the machine learning model 102 can be a recurrent neural network, a convolutional neural network, an autoencoder-based network, an autoregressive model, a contrastive self-supervised model, or a combination thereof. A transformer architecture can refer to a deep learning neural network-based design that utilizes a self-attention mechanism and is trained on autoregressive tasks to predict the next bit of its sequential or ensuing training data based on previous bits of the training data. Transformer models can be configured to capture long-range temporal dependencies, cross-channel relationships, and contextual structure in neural signal sequences. In certain embodiments, the transformer-based model can be an encoder-only model, a decoder-only model, or an encoder-decoder configuration. The transformer-based model can be adapted to operate on time-series neural data rather than text-based data.

In other embodiments, the machine learning model 102 can be a recurrent neural network (RNN)-based model. The RNN-based model can have a recurrent architecture such as long short-term memory (LSTM) networks, gated recurrent units (GRUs), or other sequence models configured to learn temporal structure in neural signals. These models can be used alone or in combination with other architectures to model sequential dependencies in neural activity.

In further embodiments, the machine learning model 102 can be a convolutional neural network (CNN)-based temporal model. The CNN-based temporal model can nave a convolutional architecture adapted for time-series analysis. Examples of such CNN-based temporal models can comprise temporal convolutional networks (TCNs) or spatiotemporal convolutional models. These models can learn local temporal, spatial, or frequency-domain patterns in neural signals and can be stacked or combined with sequence models for hierarchical representation learning.

In additional embodiments, the machine learning model 102 can be an autoencoder model or a variational autoencoder (VAE) model. In these embodiments, the machine learning model 102 can comprise an autoencoder or variational autoencoder configured to learn compressed latent representations of neural signals. Such models can be trained using reconstruction-based, predictive, or contrastive objectives and can serve as a foundation for subsequent post-training to downstream tasks.

In other embodiments, the machine learning model 102 can be a predictive coding and autoregressive model. In these embodiments, the machine learning model 102 can be implemented using architectures configured for predictive or autoregressive learning in which the model is trained to predict future segments of neural signals based on past segments. These models can learn latent representations that capture temporal dynamics and underlying structure in neural activity.

In further embodiments, the machine learning model 102 can be a contrastive and self-supervised representation learning model. In these embodiments, the machine learning model 102 can employ contrastive learning, masked prediction, or other self-supervised techniques to learn representations that distinguish between different neural signal contexts, time windows, or subjects without explicit labels.

In additional embodiments, the machine learning model 102 can comprise a hybrid architecture that combines two or more of the above approaches. For example, the machine learning model 102 can comprise convolutional layers for local feature extraction followed by transformer layers for long-range dependency modeling. The machine learning model 102 can also comprise an autoencoder-based model coupled with a sequence model for temporal prediction.

Although many embodiments described herein reference neural network-based architectures, including transformer-based machine learning models and other deep learning models, it should be understood by one of ordinary skill in the art that the disclosed systems and methods are not limited to neural networks or parametric models. In some embodiments, the machine learning model used during pre-training, post-training, inference, or downstream mapping can comprise non-parametric or classical machine learning methods, either alone or in combination with parametric models.

By way of example and without limitation, such alternative implementations can include statistical models, kernel-based methods, nearest-neighbor methods, probabilistic graphical models, linear or nonlinear regression models, support vector machines, clustering algorithms, dimensionality reduction techniques, state-space models, Bayesian inference models, hidden Markov models, Kalman filters, Gaussian processes, or other classical or non-parametric learning techniques capable of operating on neural signals, latent representations, brain-state embeddings, or contextual representations.

In some embodiments, non-parametric or classical models can operate directly on neural signals, on features derived from neural signals, or on latent representations or brain-state embeddings produced by a pretrained neural foundation model. In other embodiments, such models can be used as downstream mapping modules, decision layers, arbitration modules, or control-selection modules that consume brain-state-conditioned control information generated by a neural foundation model.

In further embodiments, hybrid architectures can be employed in which parametric neural models are used to learn task-agnostic latent representations, while non-parametric or classical models are used to perform inference, classification, clustering, prioritization, gating, or control selection based on those representations. Such hybrid configurations can provide advantages in interpretability, computational efficiency, data efficiency, or robustness, while preserving the scalability benefits of task-agnostic representation learning described herein.

Accordingly, references to a “machine learning model,” “neural foundation model,” or “post-trained machine learning model” should be understood to encompass any parametric or non-parametric machine learning technique, including classical or statistical methods, that is capable of learning structure from neural signals, aligning neural activity with contextual information, inferring brain states, or generating control information for downstream system behavior.

In certain implementations, the machine learning model 102 can be trained across neural signals collected or recorded from a plurality of subjects 114 to learn shared latent structure in neural activity, while remaining adaptable to individual subjects 114 or users 122 during post-training.

In some embodiments, pre-training the machine learning model 102 can further comprise intentionally excluding any task labels associated with the sequences of neural signals 112 when providing the sequences of neural signals 112 as inputs to the machine learning model 102. Pre-training the machine learning model 102 can be performed without requiring that the machine learning model 102 output any task identifiers, command labels, or device control actions.

The task-agnostic latent representations can be internal representations learned by the machine learning model 102 that encode structure, patterns, and relationships present in the sequences of neural signals recorded from the one or more subjects 114 without relying on any task-specific labels or predefined outputs. The task-agnostic latent representations can be distinct from raw neural signals, device control commands, or task-specific labels. Latent representations can be transformed representations of neural activity that capture salient temporal, spatial, spectral, and cross-channel features of the neural signals in a lower-dimensional or abstracted representational space. These representations are learned during unsupervised or self-supervised pre-training and are task-agnostic, meaning they are learned prior to associating the neural signals with any particular task, intent, or device control function. Latent representations can be expressed as vectors, tensors, embeddings, activation patterns, or other internal model states. Latent representations are structured such that they can later be adapted, fine-tuned, or mapped to downstream outputs such as inferred brain states, intent representations, or control information 134.

In some embodiments, the sequences of neural signals 112 can be recorded or collected continuously rather than in a rigid sequence or manner. This recording can be ongoing and neural data can be collected over time from multiple subjects 114.

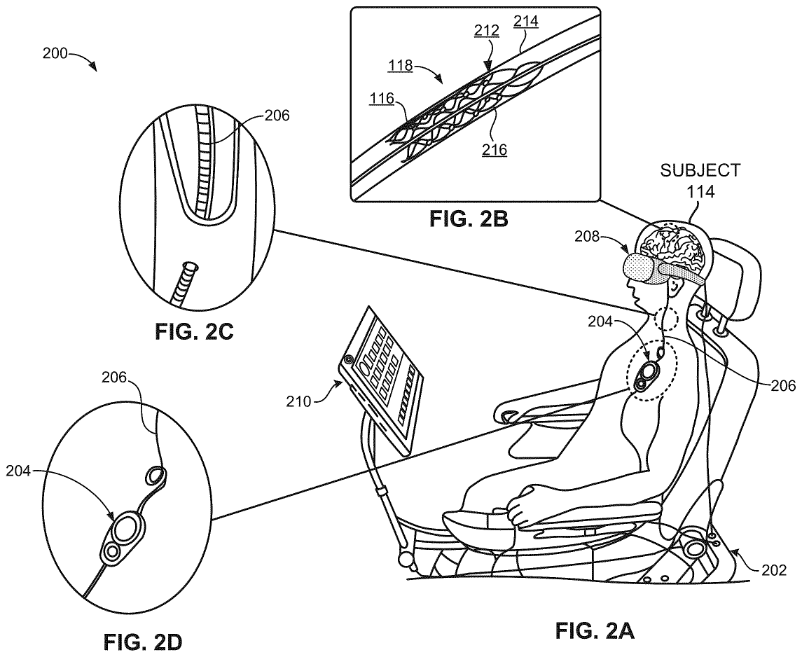

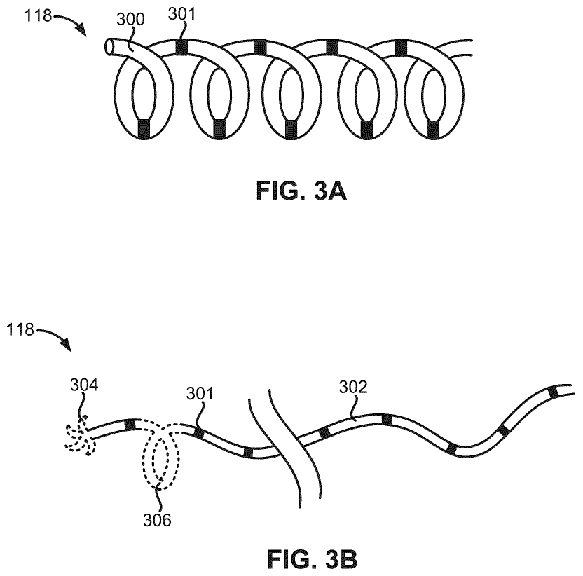

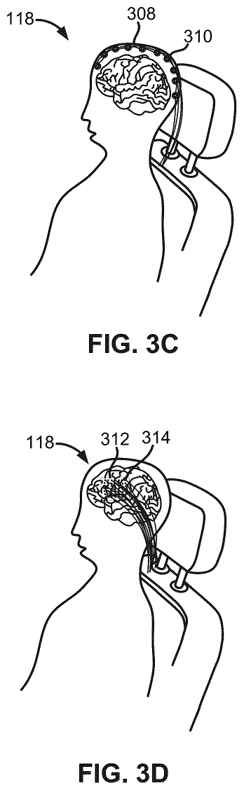

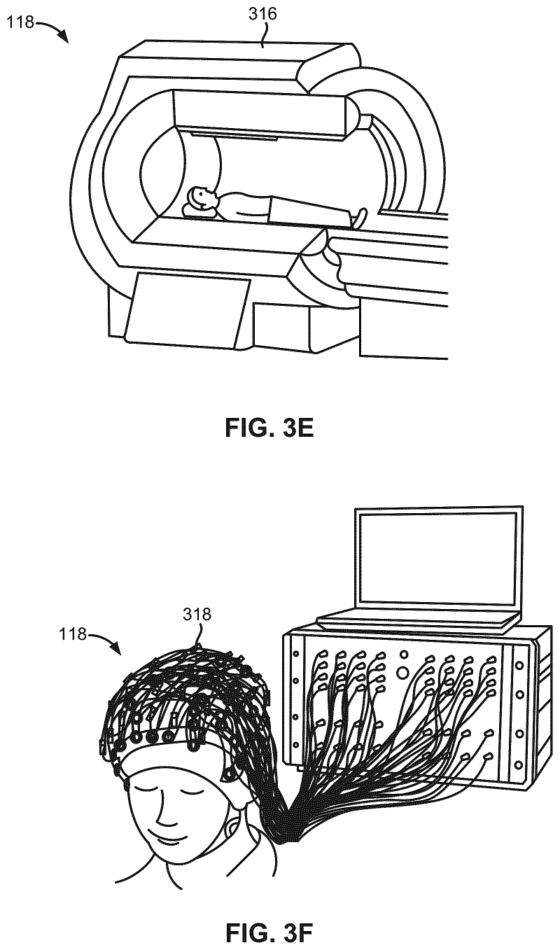

In some embodiments, the sequences of neural signals 112 can be recorded from electrodes 116 implanted within the one or more subjects 114 (see, also, e.g., FIGS. 2B and 3D). The electrodes 116 can be embedded or otherwise coupled to a recording device 118 implanted within the one or more subjects 114. For example, the electrodes 116 can be implanted endovascularly, cortically, or subcortically within the brain of each of the one or more subjects 114. As a more specific example, the sequences of neural signals 112 can be recorded from within the brain of the subject(s) 114, locations along a surface of the brain of the subject(s) 114, locations exterior to brain vessels within the brain of the subject(s) 114, locations or spaces within a dura mater of the subject(s) 114, or a combination thereof.

In other embodiments, the sequences of neural signals 112 can be recorded from the one or more subjects 114 non-invasively. For example, the sequences of neural signals 112 can be recorded from the one or more subjects via electrodes positioned or otherwise placed on the head of a subject 114 (see, e.g., FIG. 3C).

In some embodiments, the sequences of neural signals 112 can comprise at least one of neural signals that are temporally contiguous, temporally aligned, spatially aligned, and aligned by frequency. The neural signals recorded from the one or more subjects 114 can comprise raw neural signals, transient oscillatory or pseudo-oscillatory bursts or burst features, neural signal spikes, binarized neural signals, action potentials, event-related potentials, graded potentials, local field potentials, rhythmic or repetitive patterns of neural signals, chunks of neural signals, or a combination thereof recorded across different recording channels, frequencies, and time.

The sequences of neural signals 112 can refer to ordered collections of neural signal data that preserve structure along one or more dimensions relevant to neural activity. Such sequences can be constructed to reflect temporal progression, spatial distribution, frequency characteristics, or combinations thereof, and are not limited to a single representation or modality.

In some embodiments, the sequences of neural signals 112 can comprise temporal neural data, in which neural signals are ordered according to time. Temporal sequences can include contiguous or overlapping time windows of neural recordings, discrete time steps, event-based segments, or rolling buffers of neural activity. Temporal neural data can capture dynamics such as signal evolution, transient events, oscillatory patterns, or temporal dependencies between neural activations across time.

In other embodiments, the sequences of neural signals 112 can comprise spatial neural data, in which neural signals are ordered or structured according to spatial relationships among recording locations. Spatial neural data can reflect signals recorded from multiple electrodes, channels, brain regions, or anatomical locations, and can preserve information about relative position, proximity, or functional connectivity between recording sites. Spatial sequencing can occur alone or in combination with temporal ordering.

In some embodiments, the sequences of neural signals 112 can comprise frequency neural data, in which neural signals are represented in terms of spectral content, frequency bands, or time-frequency decompositions. Frequency neural data can include representations derived from transforms such as Fourier transforms, wavelet transforms, filter banks, or other spectral analyses, and can capture relationships between low-frequency and high-frequency components, cross-frequency coupling, or band-specific activity.

The sequences of neural signals 112 can also comprise combinations of temporal, spatial, and frequency neural data. For example, a sequence of neural signals 112 can represent time-ordered neural activity across multiple electrodes 116 and frequency bands, forming a multidimensional representation that preserves temporal progression, spatial structure, and spectral characteristics simultaneously. Such a sequence of neural signals 112 can be represented as vectors, matrices, tensors, or other structured data formats suitable for input to the machine learning model 102.

In these embodiments, the ordering, segmentation, and representation of the sequences of neural signals 112 can vary depending on implementation and do not require a particular sampling rate, window length, spatial resolution, or frequency decomposition. This disclosure is not limited to a specific method of constructing such sequences, provided that the sequences retain sufficient structure to enable unsupervised or self-supervised learning of latent representations from neural activity.

In some embodiments, each chunk of raw neural signals can be a 10 ms recording of raw neural signals. Each chunk of raw neural signals (e.g., a 10 ms recording) can be considered a token or discrete unit that can be provided as inputs to the machine learning model 102. This means that a neural signal recording lasting only a few seconds (e.g., 3 seconds to 5 seconds) can yield thousands of tokens or discrete units across the various electrodes 116 of the recording device 118 and across the various desired frequency bands.

In some embodiments, a pre-processing module running on a control unit 202 (see, e.g., FIG. 2A) or one or more servers 120 can filter the raw neural signals recorded from the recording device 118 in one or more desired frequency bands using one or more bandpass filters, wavelet convolutions, or a combination thereof. The pre-processing module can also convert voltage values of the filtered raw neural signals into power values (expressed as V²/Hz or μV²/Hz, dB/Hz, S² where S denotes the units of the signal, etc.) or normalized power values (expressed as z-scores, ratios, differences, percentage changes).

The pre-processing module can also apply at least one of a power threshold and a duration threshold for each of the desired frequency bands. The power threshold and/or the duration threshold can be selected or optimized for each subject 114.

In some embodiments, the desired frequency bands can be between 0.1 Hz and 32 kHz. The desired frequency bands can also be between 4 Hz and 400 Hz. In certain embodiments, the desired frequency bands can be between 20 Hz and 200 Hz. In other embodiments, the desired frequency bands can be between 35 Hz and 150 Hz.

In some embodiments, the pre-processing module can apply at least one of a power threshold and a duration threshold to identify or detect a number of transient oscillatory or pseudo-oscillatory bursts from the neural signals recorded. For example, the pre-processing module can identify or detect the transient oscillatory or pseudo-oscillatory bursts in response to one of the magnitude or power-related values exceeding the power threshold and/or the duration threshold for each of the desired frequency bands.

The transient oscillatory or pseudo-oscillatory bursts can also be referred to as “transients,” “oscillation events,” “band-bursts (e.g., beta-bursts),” “band-events (e.g., gamma-events),” “miniature evoked responses,” or “oscillatory bursts.” The oscillatory or pseudo-oscillatory bursts can be characterized by being transient, meaning that each burst lasts for only a very short duration and that each burst is a high-energy burst, meaning that the power of each burst exceeds a threshold power level determined relative to a baseline level of neural activity and/or background noise.

In some embodiments, the duration of a typical transient oscillatory or pseudo-oscillatory burst can last between 1 ms to 100 ms. In other embodiments, the duration of a typical transient oscillatory or pseudo-oscillatory burst can last between 10 ms and 100 ms. The duration of a transient oscillatory or pseudo-oscillatory burst can depend on factors such as a frequency-band measured. For example, the transient oscillatory or pseudo-oscillatory burst can last between 1 ms to 10 ms when the frequency-band measured is relatively high (e.g., gamma-band) or last greater than 10 ms when the frequency-band measured is lower (e.g., alpha-band).

For example, the transient oscillatory or pseudo-oscillatory bursts can be referred to as beta bursts or beta-band bursts if these bursts were obtained from signals in the beta-oscillatory band (having a frequency of approximately 15-35 Hz). In addition, the transient oscillatory or pseudo-oscillatory bursts can be referred to as gamma bursts or gamma-band bursts if these bursts were obtained from signals in the gamma-oscillatory band (having a frequency of approximately 45-100 Hz). Moreover, the transient oscillatory or pseudo-oscillatory bursts can be referred to as alpha bursts or alpha-band bursts if these bursts were obtained from signals in the alpha-oscillatory band (having a frequency of approximately 7 Hz to 12 Hz). Furthermore, the transient oscillatory or pseudo-oscillatory bursts can be referred to as theta bursts or theta-band bursts if these bursts were obtained from signals in the theta-oscillatory band (having a frequency of approximately 4 Hz to 7 Hz).

The pre-processing module can extract one or more burst features from the transient oscillatory or pseudo-oscillatory bursts detected within a predetermined or preset detection period. The pre-processing module can detect upwards of hundreds of transient oscillatory or pseudo-oscillatory bursts within each detection period across the various electrodes 116 of the recording device 118 and across the various frequency bands (e.g., 0.1 Hz to 32 kHz).

In some embodiments, the detection period can be between 10 milliseconds (ms) and 100 ms. More specifically, the detection period can be between 50 ms and 100 ms. For example, the detection period can be about 100 ms.

The pre-processing module can extract the one or more burst features by counting or summing the number of transient oscillatory or pseudo-oscillatory bursts detected and determining the timing of such bursts. The pre-processing module can also determine the frequency, power value, and duration of each burst.

The burst features can comprise a burst count, a burst rate, a burst band frequency or frequency distribution, an interburst interval length (single channel and across multiple channels), a burst timing or timing pattern, an average burst duration, a burst waveform (e.g., the time domain waveform of a burst), or any changes or combination thereof. The burst features can also comprise an average power across bursts within a window of time, a maximum power of the bursts, a number of cycles, a peak frequency of the bursts, a minimum frequency of the bursts, a maximum frequency of the bursts, a frequency span (expressed in octaves), an average power just before and/or just after a burst, a low-frequency instantaneous phase at the time of a high-frequency burst, alpha and beta power at the time of a high-frequency burst, an oscillatory score (i.e., a correlation between the filtered and raw signal at the time of a burst). The burst features can also comprise a burst synchronization or distance (i.e., a measure of the correlation between bursts at different channels when treated as a point process), the left and/or right slope of the transient bursts (i.e., how fast does the amplitude rise or fall), and repeating sequences in time of transient bursts (e.g., certain user thoughts or movement types can generate a sequence of bursts that appear at certain electrodes at specific time intervals).

In additional embodiments, the sequences of neural signals 112 can be recorded from the one or more subjects 114 via an imaging modality such as functional magnetic resonance imaging (fMRI) (see, e.g., FIG. 3E). In further embodiments, the sequences of neural signals 112 can be recorded from the one or more subjects 114 via functional near infrared spectroscopy (see, e.g., FIG. 3F).

In some embodiments, pre-training the machine learning model 102 can further comprise providing the sequences of neural signals 112 recorded from the one or more subjects 114 as inputs to the machine learning model 102 and obtaining, as outputs, autoregressive predictions, masked neural signal predictions, or contrastive learning predictions of upcoming or ensuing neural signals.

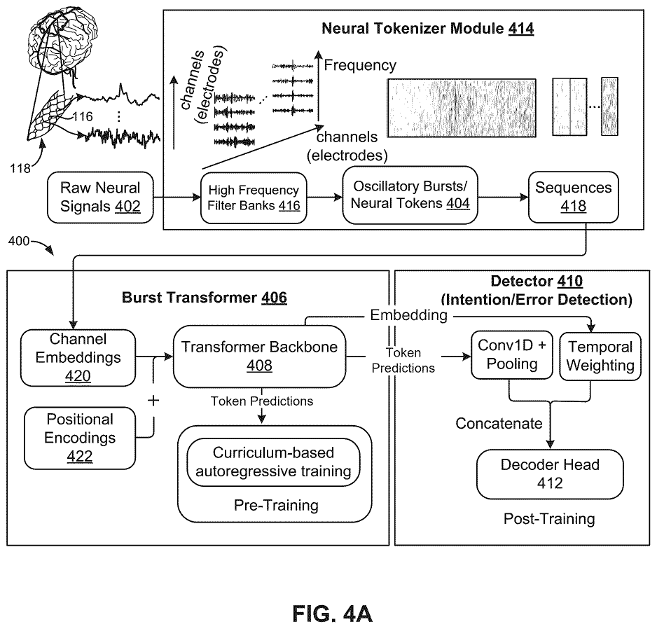

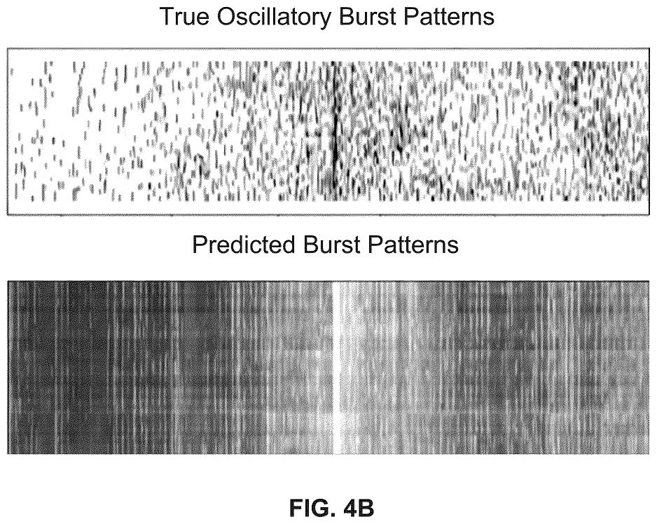

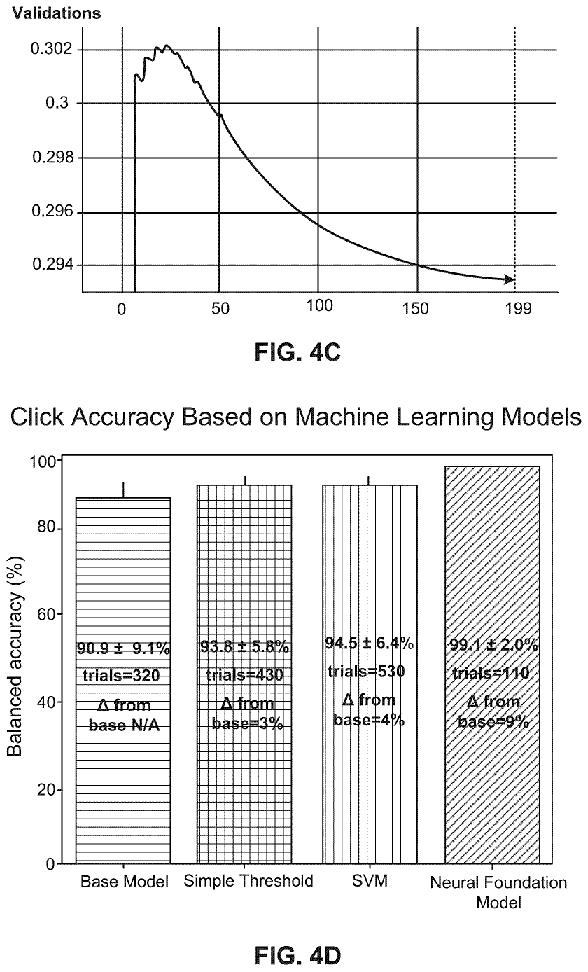

In some embodiments, the sequences of neural signals 112 provided as inputs to the machine learning model 102 can be provided as discretized neural signals treated as tokenized inputs (see, e.g., FIG. 4A).

In certain embodiments, pre-training the machine learning model 102 in an autoregressive manner can refer to training the machine learning model 102 to predict one or more portions of a sequence of neural signals 112 based on preceding portions of the sequence. Rather than relying on externally supplied labels or predefined tasks, the machine learning model 102 can use the structure inherent in the neural signal data itself as a supervisory signal. For example, given a sequence of neural signal segments, the machine learning model 102 can be trained to predict a subsequent segment, a masked segment, or a future time window of neural activity from earlier segments. To enable such autoregressive learning, discretized neural activity can be represented as tokenized inputs, analogous to how words, sub-words, or characters are represented as tokens in language models. In this context, a “token” does not imply linguistic meaning, but instead represents a discrete unit of neural information derived from neural signals, such as a time window, channel-specific segment, frequency-band component, event-based segment, or combination thereof. These tokenized inputs allow neural signal sequences to be modeled as ordered series of discrete units suitable for sequence-based learning.

By training the machine learning model 102 to operate on tokenized neural sequences and to predict portions of those sequences autoregressively, the machine learning model 102 can learn latent structure present in neural activity, including temporal dependencies, cross-channel relationships, and recurring neural patterns. Importantly, this learning occurs prior to defining any task-specific or intent-specific labels, enabling the machine learning model 102 to acquire general, task-agnostic representations of neural activity that can later be adapted during a post-training phase to support a wide range of downstream inference or device control tasks.

Pre-training the machine learning model 102 can be done in an unsupervised or self-supervised manner. As shown in FIG. 1A, the output of the pre-training phase can be a pre-trained machine learning model 104.

Pre-training the machine learning model 102 can further comprise optimizing the machine learning model 102 using certain optimization techniques such as stochastic gradient descent. Also, for example, other optimization techniques or algorithms can be used including the Adam optimization technique and/or the AdamW optimization technique.

In certain alternative embodiments, the machine learning model 102 can also be pre-trained on unlabeled contextual information or raw contextual information. In these embodiments, the unlabeled contextual information or raw contextual information can be provided along with the unlabeled sequences of neural signals 112.

In some embodiments, pre-training the machine learning model 102 can further comprise co-training the machine learning model 102 with textual information temporally aligned with the sequences of neural signals 112. The textual information can provide human-interpretable semantic references or descriptions of events, environments, stimuli, or tasks occurring at or around the time the neural signals are recorded that can be used to ground, probe, or validate learned latent neural representations without directly supervising model outputs. Such textual information is not used as task labels or direct supervisory outputs but instead serves as an auxiliary modality that provides semantic context for evaluating and shaping the learned neural representations.

In some cases, a purely autoregressive neural pre-training objective (e.g., predicting the next segment of neural activity from prior segments) can achieve low predictive loss while still learning latent neural representations that are difficult to interpret or validate. In other words, a machine learning model 102 can become very good at predicting “more neural signals” without learning structure that corresponds to meaningful cognitive or behavioral concepts. Co-training with textual information addresses this technical problem by providing a human-interpretable semantic reference that can be aligned with neural activity. This can provide insights as to whether the machine learning model 102 is learning latent neural representations that correspond to real-world meaning rather than merely signal dynamics.

Textual information can serve one or more of the following roles including semantic anchoring of neural representations, probing and validation of latent spaces, and cross-modal alignment with other foundation models. With respect to semantic anchoring of neural representations, textual information can provide a medium in which semantic relationships are already well structured (e.g., “house” and “building” are semantically related). By examining whether neural latent representations associated with semantically related text cluster or align, assessments can be made as to whether the machine learning model 102 is learning cognitively meaningful structure. With respect to probing and validation of latent spaces, textual information can enable probing of the model's latent neural representations during training or evaluation, helping determine whether improvements in predictive loss correspond to meaningful representational learning rather than overfitting to signal statistics. With respect to cross-modal alignment with other foundation models, textual information can act as a bridge between neural foundation models and other foundation models (e.g., language or vision models). By comparing or aligning latent representations across modalities, the machine learning model 102 can be assessed for convergence toward shared abstract representations, without requiring the model to generate text as an output.

Examples of textual information can comprise, but are not limited to, sentences or words that subject(s) 114 are reading, captions or descriptions of scenes that the subject(s) 114 are viewing, textual descriptions of tasks being performed by the subject(s) 114, symbolic or natural-language descriptions of the environment surrounding the subject(s) 114, and logs or annotations describing ongoing activities undertaken by the subject(s) 114. Such textual information can be time-aligned or temporally aligned with the sequences of neural signals 112 but need not be exhaustive or precise.

In some embodiments, the co-training can be implemented in various ways including processing the textual information using a separate machine learning model and comparing the textual information to neural latent representations. The co-training can also be implemented by embedding the textual information into a shared or comparable latent space. In certain embodiments, alignment metrics, probes, or auxiliary objectives can be used to evaluate or encourage semantic correspondence. In these embodiments, the machine learning model 102 is not required to output any text.

As shown in FIG. 1A, in some embodiments, the machine learning model 102 can be run on one or more servers 120 in a cloud computing environment 121 (i.e., in the cloud). In these embodiments, the machine learning model 102 can be pre-trained in the cloud.

In some embodiments, the one or more servers 120 can comprise or refer to one or more virtual servers or virtualized computing resources. For example, the servers 120 can refer to virtual servers or cloud servers hosted and delivered by a cloud computing platform (e.g., Amazon Web Services®, Microsoft Azure®, or Google Cloud®). In other embodiments, the one or more servers 120 can refer to one or more stand-alone servers such as a rack-mounted server, a blade server, a mainframe, a dedicated desktop or laptop computer, one or more processors or processor cores therein, or a combination thereof.

In some embodiments, each of the servers 120 can comprise one or more server processors, server memory and storage units, and a server communication interface. The server processors can be coupled to the server memory and storage units and the server communication interface through high-speed buses or interfaces.

The one or more server processors can comprise one or more central processing units (CPUs), graphics processing units (GPUs), application-specific integrated circuits (ASICs), field-programmable gate arrays (FPGAs), or a combination thereof. The one or more server processors can execute software stored in the server memory and storage units to execute the methods or instructions described herein. The one or more server processors can be embedded processors, processor cores, microprocessors, logic circuits, hardware finite-state machines (FSMs), digital signal processors (DSPs), or a combination thereof. The one or more server processors can be configured to run the machine learning models disclosed herein.

The server memory and storage units can store software instructions, data (including video or image data), tables, logs, databases, or a combination thereof. The server memory and storage units can comprise an internal memory and/or an external memory, such as a memory residing on a storage node or a storage server. The server memory and storage units can be a volatile memory or a non-volatile memory. For example, the server memory and storage units can comprise nonvolatile storage such as NVRAM, Flash memory, solid-state drives, hard disk drives, and volatile storage such as SRAM, DRAM, or SDRAM.

The server communication interface can refer to one or more wired and/or wireless communication interfaces or modules. For example, the server communication interface can be a network interface card. The server communication interface can comprise or refer to at least one of a WiFi communication module, a cellular communication module (e.g., a 4G or 5G cellular communication module), and a Bluetooth®/BLE or other type of short-range communication module.

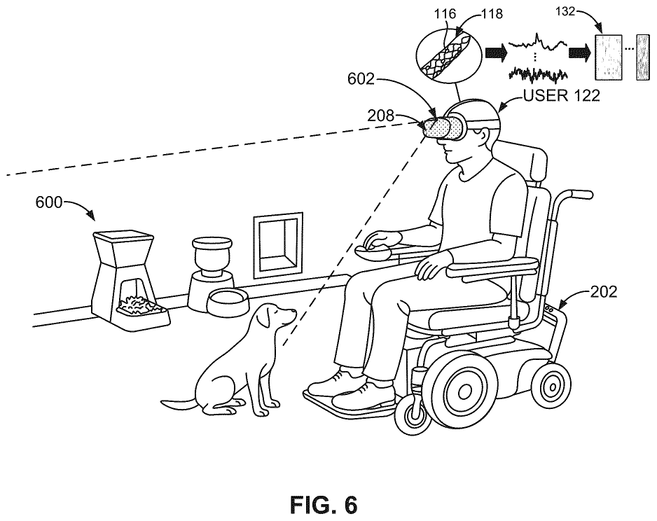

Each of the servers 120 can connect to or communicatively couple with client devices or computing devices including control unit 202 (see, e.g., FIG. 2A or FIG. 6) via the server communication interface. Each of the servers 120 can transmit data packets to or receive data packets from the control unit 202 and/or other computing devices using the server communication interface.

In some embodiments, software instructions run on the one or more servers 120, including any of the method steps or workflows disclosed herein, can be written in the Ruby® programming language, Python® programming language, Java® programming language, C programming language, C++ programming language, C #programming language, JavaScript programming language, or a combination thereof.

In other embodiments, the machine learning model 102 can be run on one or more computing devices. In these embodiments, the one or more computing devices can be communicatively coupled or otherwise connected to the control unit 202 (see, e.g., FIG. 2A or FIG. 6).

FIG. 1B illustrates that the pre-trained machine learning model 104 can be post-trained using supervised learning on the sequences of neural signals 112 recorded from the one or more subjects 114 aligned with labeled contextual information 124 to yield a post-trained machine learning model 106. In some embodiments, the sequences of neural signals 112 can be temporally aligned with the labeled contextual information 124 and both are provided as inputs to the pre-trained machine learning model 104.

In some embodiments, the sequences of neural signals 112 are recorded from subjects 114 that end up using the deployed instance of the post-trained machine learning model 106 to control devices 108 or software applications 110 running on such devices 108. In these embodiments, such subjects 114 can also be considered users 122 (see, e.g., FIG. 1C).

In additional embodiments, users 122 can also refer to individuals that did not participate in the pre-training phase. In these embodiments, additional sequences of neural signals 112 were recorded from such users 122 and these additional sequences of neural signals 112 were only used to post-train the pre-trained machine learning model 104 but not for pre-training the machine learning model 102 as part of the unsupervised or self-supervised learning phase. As such, it should be understood by one of ordinary skill in the art that even though FIG. 1B depicts sequences of neural signals 112 recorded from subject(s) 114, the post-training phase can also comprise recording sequences of neural signals 112 from user(s) 122 that end up using the deployed instance of the post-trained machine learning model 106 to control devices 108 or software applications 110 running on such devices 108 (see, e.g., FIG. 1C). In these embodiments, post-training the pre-trained machine learning model 104 can comprise providing labeled contextual information 124 aligned with these additional sequences of neural signals 112 recorded from the user(s) 122 to the pre-trained machine learning model 104 to yield the post-trained machine learning model 106.

The labeled contextual information 124 can be obtained by recording, logging, or otherwise extracting contextual data or information using one or more modalities in real environments (e.g., using digital cameras, digital audio recording devices, and environmental sensors to record or log videos, images, or data in real life) or simulated environments (e.g., using virtual-reality (VR) headsets or augmented-reality (AR) to record or log contextual data or information in VR environments or AR environments) experienced or viewed by the one or more subjects 114 or one or more users 122.

The contextual data or information can be recorded or logged automatically using one or more sensors or instrumentation of a portable device 126 that can be worn by the subject(s) 114 or user(s) 122 or carried by the subject(s) 114 or user(s) 122. In other embodiments, the portable device 126 can accompany the subject(s) 114 or user(s) 122 such as being installed or otherwise coupled to a mobility vehicle carrying the subject(s) 114 or user(s) 122 or an ambulatory device used by the subject(s) 114 or user(s) 122. In some embodiments, the contextual data or information can be recorded or logged automatically using one or more sensors or instrumentation without any manual annotation.

The labeled contextual information 124 can comprise descriptive representations of the environments or activities of the subject(s) 114 or user(s) 122. For example, the labeled contextual information 124 can comprise labeled instances of contextual data or information concerning a real environment surrounding the subject(s) 114 or user(s) 122 or a simulated environment experienced or viewed by the subject(s) 114 or user(s) 122. For example, the labeled contextual information 124 can comprise labeled instances of contextual data or information concerning a setting, a location, a time-of-day, a day-of-the-week, a month, a year, a season, a weather condition, a detected activity, one or more objects or individuals detected within a real environment surrounding the subject(s) 114 or user(s) 122, one or more objects or individuals within the simulated environment viewed or otherwise experienced by the subject(s) 114 or user(s) 122, one or more connected devices detected, or a combination thereof.

The labeled contextual information 124 can be in the form of text labels or text-based files, semantic labels, tags, keywords, metadata, numerical labels, key-value pairs, machine-readable data structures, sensor data or sensor logs, device data or device logs, audio data or audio file(s), image data or an image file(s), and/or video data or video file(s).

The labeled contextual information 124 can comprise contextual data or information outputted by one or more object-detection machine learning models or other types of deep learning or computer vision models.

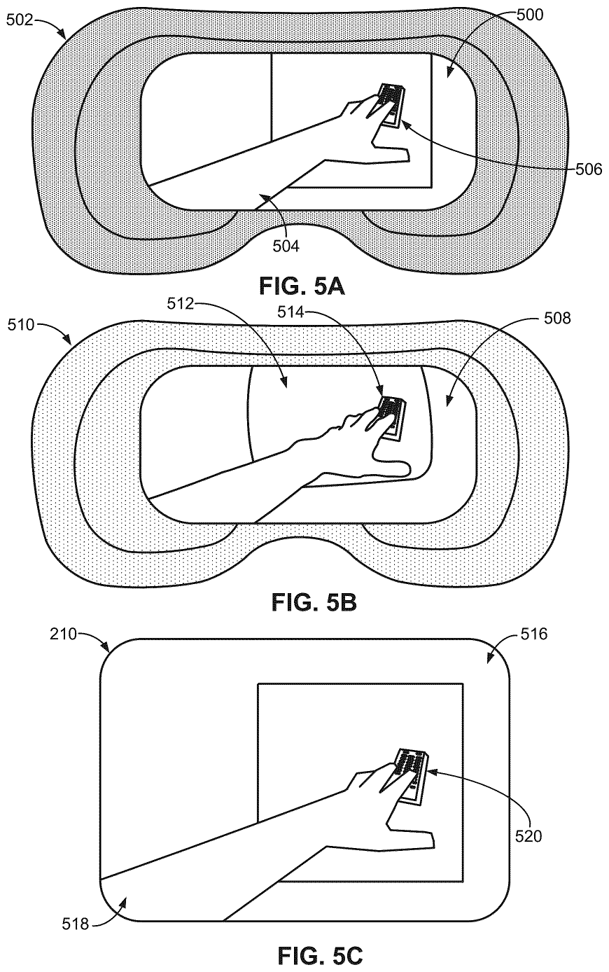

The labeled contextual information 124 can be derived or otherwise obtained by recording, logging, or otherwise extracting contextual data or information from one or more modalities. In some embodiments, the modalities can be implemented as a portable device 126 that can be worn by the subject(s) 114 or user(s) 122, carried by the subject(s) 114 or user(s) 122, or otherwise accompanying the subject(s) 114 or user(s) 122. For example, the portable device 126 can refer to a wearable display device 208 (see, e.g., FIG. 2A) such as a VR headset 502 (see, e.g., FIG. 5A) or an AR wearable 510 (see, e.g., FIG. 5B). The AR wearable 510 can be an AR headset or a pair of smart glasses. Also, for example, the portable device 126 can refer to a portable client device such as a smartphone, a tablet computer, or a laptop computer. In additional embodiments, the portable device 126 can refer to an audio/video recording device such as a sound recording device or digital video camera.

In some embodiments, the contextual information used during pre-training, post-training (e.g., the labeled contextual information 124), or real-time (e.g., the real-time contextual information 136) can be obtained from one or more wearable devices or physiological sensing devices associated with the subject(s) 114 or user(s) 122. Such devices can capture movement-related, physiological, behavioral, or attentional signals that provide additional context temporally aligned with neural signals recorded from the subject(s) 114 or user(s) 122.

By way of example and without limitation, wearable or sensing devices can include gesture-sensing gloves, motion trackers, inertial measurement units (IMUs), eye-tracking devices, pupil diameter trackers, heart rate monitors, electrocardiogram (ECG) sensors, blood oxygenation (SpO2) sensors, blood pressure monitors, galvanic skin response sensors, respiration monitors, electromyography (EMG) sensors, or combinations thereof. These devices can be worn on the hands, wrists, head, torso, limbs, or other suitable locations, or can be integrated into one or more head-mounted displays, glasses, watches, bands, or clothing.

Data obtained from such devices can serve as contextual information describing a physical action, a physiological state, an attentional focus, an arousal level, or an interaction of the subject(s) 114 or user(s) 122 with their environment and can be temporally synchronized with neural signals for use as supervisory signals during the post-training of the pre-trained machine learning model 104. For example, gesture data or motion data can provide contextual information indicating attempted or executed movements; eye tracking and pupil measurements can provide contextual information related to visual attention, cognitive load, or engagement; and physiological signals such as heart rate or blood oxygenation can provide contextual information related to stress, effort, fatigue, or emotional state.

In some embodiments, such wearable-derived contextual information can be particularly useful during longitudinal data collection, population-scale training, or environment-induced training scenarios, as they provide complementary signals that help disambiguate brain states, improve separability of latent representations, and enhance robustness of learned brain-state embeddings over time.

It should be understood by one of ordinary skill in the art that not all wearable devices are required or appropriate for all user or subject populations. For example, certain motion-based sensors or gesture-based sensors may be less applicable to subject(s) 114 or user(s) 122 with severe motor impairments or locked-in syndrome. However, other wearable or physiological sensors such as eye tracking sensors, pupil diameter tracking sensors, heart rate monitoring sensors, or blood oxygenation monitoring sensors, can remain applicable across a wide range of user or subject populations, including subject(s) 114 or user(s) 122 with limited or no voluntary motor output.

Accordingly, the systems and methods disclosed herein are not limited to any particular wearable or physiological sensing modality, and contextual information can be selectively obtained from any combination of wearable, environmental, or physiological data sources that provide time-aligned contextual signals usable to enrich training, alignment, or inference of the neural foundation model.

As shown in FIG. 1B, the sequences of neural signals 112 (e.g., spontaneous neural signals) can be passively collected or recorded from the subject(s) 114 or user(s) 122 while the subject(s) 114 or user(s) 122 go about certain activities in the real-world or passively observes an environment. In this scenario, the contextual data or information can be recorded, logged, extracted, or otherwise obtained by passively observing the activities or environment (e.g., a real environment or virtual environment) of the subject(s) 114 or user(s) 122. Passively observing the environment or the activities of the subject(s) 114 or user(s) 122 can further comprise observing the environment or the activities of the subject(s) 114 or user(s) 122 without intentionally modifying the environment of the subject(s) 114 or user(s) 122 or interfering with the activities of the subject(s) 114 or user(s) 122. For example, the portable device 126 can passively record, log, or capture sounds, videos, or images of the activities or environment of the subject(s) 114 or user(s) 122. Descriptive representations (e.g., text labels, semantic labels, tags, keywords, metadata, etc.) of the activities or the environment can then be determined or otherwise obtained from such sounds recordings, videos, and/or images. At the same time that the contextual data or information is being passively recorded, logged, or captured, the sequences of neural signals 112 of the subject(s) 114 or user(s) 122 can also be passively collected or recorded. Moreover, as shown in FIG. 1B, the sequences of neural signals 112 passively collected or recorded from the subject(s) 114 or user(s) 122 and the contextual data or information passively recorded, logged, or captured can be stored in an aggregated dataset 130.

Passively observing an environment or activities of the subject(s) 114 or user(s) 122 refers to acquiring contextual data or information about what is occurring without intentionally intervening to change the neural condition or behavior of the subject(s) 114 or user(s) 122 at that time. In this mode, neural signals are recorded along with contextual data or information that naturally arises from the ongoing experience of the subject(s) 114 or user(s) 122.

The key characteristics of this observation can include no deliberate task imposition or experimental manipulation, no requirement that a particular brain state be elicited, context is captured as it naturally occurs, and data collection can be continuous and longitudinal.

Some examples of passively observing the environment or activities of the subject(s) 114 or user(s) 122 can include recording neural signals while the subject(s) 114 or user(s) 122 goes about daily activities in a physical environment (e.g., home, workplace, etc.), capturing video, audio, text logs, or sensor data describing what the subject(s) 114 or user(s) 122 is seeing, hearing, or interacting with, observing virtual or augmented environments that the subject(s) 114 or user(s) 122 is already using (e.g., a computer interface or AR display) without directing a specific task, recording perceptual stimuli (visual scenes, sounds) correlated with neural activity, or logging inferred internal brain activity patterns that occur naturally, without inducing them. Passive observation provides broad, scalable contextual coverage that enables large-scale data collection across time and subject(s) 114 or user(s) 122, captures naturally occurring neural variability, and supports unsupervised or context-aligned learning without task constraints.

In some embodiments, the aggregated dataset 130 can refer to one or more databases stored on or accessible by the one or more servers 120 in the cloud or stored on or accessible by the control unit 202 (see, e.g., FIG. 2A). In some embodiments, the aggregated dataset 130 can store unlabeled neural data, neural data with labels or labeled neural data, and neural data aligned (e.g., temporally aligned) with labeled contextual information 124. As part of the post-training phase, the appropriate subset of this aggregated dataset 130 can be selected and a supervised learning objective or a context-supervised learning objective can be applied to the pre-trained machine learning model 104.

Additionally, or alternatively, as shown in FIG. 1B, sequences of neural signals 112 can also be collected in a task-driven or intentional manner. In these embodiments, the environment (e.g., an AR environment or a VR environment) of the subject(s) 114 or user(s) 122 can be modified, modulated, or adjusted in order to induce the subject(s) 114 or user(s) 122 to generate, conjure, or invoke targeted neural activity. For example, the portable device 126, when implemented as a VR headset 502 or AR wearable 510, can modify at least part of a simulated environment (e.g., a VR environment or AR environment) experienced or viewed by the subject(s) 114 or user(s) 122 to induce the subject(s) 114 or user(s) 122 to generate, conjure, or invoke certain targeted neural activity. The sequences of neural signals 112 can be collected or recorded from the subject(s) 114 or user(s) 122 while the subject(s) 114 or user(s) 122 experiences or views the modified environment (e.g., the modified VR environment or the modified AR environment). For example, such modifications can include displaying or adjusting one or more rendered objects or rendered individuals or animals to the subject(s) 114 or user(s) 122, displaying or adjusting one or more new settings or scenery to the subject(s) 114 or user(s) 122, or allowing the subject(s) 114 or user(s) 122 to interact with such rendered objects, individuals, animals, or settings.

The portable device 126 can passively record, log, or capture sounds, videos, or images of the modified environments experienced or viewed by the subject(s) 114 or user(s) 122. The portable device 126 or one or more computing devices controlling the portable device 126 or communicatively coupled to the portable device 126 can record or log descriptive representations (e.g., text labels, semantic labels, tags, keywords, metadata, etc.) of the modified environments (e.g., modified VR environment or modified AR environment) or the rendered objects, individuals, animals, or settings within such environments. Intentionally modifying the environment of the subject(s) 114 or user(s) 122 will also be discussed in more detail in relation to FIGS. 5A and 5B.

In some embodiments, modifying the environment of the subject(s) 114 or user(s) 122 can refer to intentionally altering the environment or experience(s) of the subject(s) 114 or user(s) 122 to cause the subject(s) 114 or user(s) 122 to induce or elicit particular neural conditions or brain states that may be underrepresented or difficult to observe through passive recording alone. The key characteristics of such modifications can include deliberately structuring or manipulating an environment of the subject(s) 114 or user(s) 122 using the portable device 126 or another device. This intervention is active and intentional and the goal is to reliably evoke specific brain states or underrepresented or difficult to observe brain states.

Some examples of modifying the environment of the subject(s) 114 or user(s) 122 can include presenting or otherwise displaying a VR scenario to the subject(s) 114 or user(s) 122 designed to elicit motor planning, decision-making, or stress responses; using AR or extended reality overlays to guide attention or perception, structuring interactive tasks that provoke error detection, anticipation, or goal-directed behavior; modifying sensory input (e.g., visual or auditory inputs) to evoke targeted neural responses; and adjusting environmental parameters (e.g., timing, difficulty, stimuli, etc.) to amplify signal separability.

One technical advantage of intentionally modifying the environment of the subject(s) 114 or user(s) 122 is that the pre-trained machine learning model 104 is able to learn much faster during the post-training phase, the signal-to-noise ratio for specific brain states is increased, and supervisory contexts can be generated in instances where passive data is insufficient. By intentionally modifying the environment of the subject(s) 114 or user(s) 122, precision and controllability are prioritized over continuous coverage.

At the same time that the contextual data or information is being recorded, logged, or captured, the sequences of neural signals 112 of the subject(s) 114 or user(s) 122 experiencing or viewing the modified environment(s) can also be collected or recorded. Moreover, as shown in FIG. 1B, the sequences of neural signals 112 collected or recorded from the subject(s) 114 or user(s) 122 and the contextual data or information recorded, logged, or captured can be stored in the aggregated dataset 130. In some embodiments, the contextual data or information recorded, logged, or otherwise captured can be enriched with cognitive primitives.

Also, as shown in FIG. 1B, the sequences of neural signals 112 (passively collected as spontaneous neural signals or actively induced) can be temporally aligned (e.g., synchronized in time) with the labeled contextual information 124 and both provided as inputs to the pre-trained machine learning model 104. During the post-training, the pre-trained machine learning model 104 can transform task-agnostic latent representations learned during the pre-training phase (see, e.g., FIG. 1A) into brain-state embeddings configured to align neural and contextual representations.

In some embodiments, post-training the pre-trained machine learning model 104 can further comprise inputting the sequences of neural signals 112 into the pre-trained machine learning model 104 and obtaining, as outputs, predictions concerning a context or contextual information based on the neural signals inputted.

In certain embodiments, the post-training operates on latent representations learned during the pre-training phase rather than raw neural signals alone. Labels, control actions, or context-derived supervisory signals are applied after the model has learned task-agnostic representations. This dramatically reduces the amount of labeled contextual information 124 required per subject 114 or user 122. This also support scalability since small subject-specific datasets can be used to adapt a pre-trained machine learning model 104 trained at a population level.

In some embodiments, the pre-trained machine learning model 104 and the post-trained machine learning model 106 can be run on one or more servers 120 in a cloud computing environment 121 (i.e., in the cloud). In these embodiments, the pre-trained machine learning model 104 can be post-trained in the cloud.

As will be discussed in more detail in the following sections, the sequences of neural signals 112 can be recorded by a recording device 118. In embodiments where the recording device 118 is an implantable device, the recording device 118 can be connected to a telemetry unit 204 (see, e.g., FIGS. 2A and 2D) that is communicatively coupled (e.g., via wireless or wired connections) to a control unit 202 (see, e.g., FIG. 2A). In other embodiments, the recording device 118 can be communicatively coupled directly to the control unit 202 or to another computing device.

In some embodiments, the control unit 202 can be communicatively coupled to the one or more servers 120 in the cloud. In these embodiments, the control unit 202 can store the sequences of neural signals 112 in the aggregated dataset 130 and eventually transmit the sequences of neural signals 112 to the one or more servers 120 in order to input the sequences of neural signals 112 to the pre-trained machine learning model 104.

In some embodiments, the control unit 202 can also be communicatively coupled to a wearable display device 208 worn by the subject(s) 114 or user(s) 122 or another type of portable device 126 carried by the subject(s) 114 or user(s) 122 or within a vicinity of the subject(s) 114 or user(s) 122, or a device or server controlling the wearable display device 208 or the portable device 126. In these embodiments, at least some of the labeled contextual information 124 derived from the contextual data or information recorded, logged, or captured by or otherwise obtained from the wearable display device 208 and/or another type of portable device 126 can be transmitted from the control unit 202 to the one or more servers 120.

In other embodiments, the one or more servers 120 can retrieve or otherwise obtain the contextual data or information directly from the wearable display device 208 worn by the subject(s) 114 or user(s) 122 or another type of portable device 126 carried by the subject(s) 114 or user(s) 122 or accompanying the subject(s) 114 or user(s) 122.

FIG. 1C illustrates a user 122 using the post-trained machine learning model 106 to control one or more devices 108 or software applications 110 running on such devices 108. The method 100C can comprise inputting real-time neural signals 132 recorded from the user 122 into the post-trained machine learning model 106 to generate control information 134. The method 100C can also comprise controlling the one or more devices 108 or software applications 110 running on such devices 108 based on the control information 134.

It should be understood by one of ordinary skill in the art that even though the compound word “real-time” is used in reference to real-time neural signals 132 and, later, to real-time contextual information 136, the signals or information referenced by such terms can be recorded, captured, or otherwise collected in near-real-time or after a short (e.g., several seconds or several milliseconds) delay.

In some embodiments, the post-trained machine learning model 106 can be configured for real-time use by structurally and functionally decoupling representation learning from task execution, such that neural signals recorded from user(s) 122 during operation are processed through a model that has already internalized general neural structure during the pre-training phase and subsequently adapted to produce actionable outputs. Unlike task-first or decoder-centric systems, the post-trained machine learning model 106 can operate by reusing latent representations learned across neural signal sequences and applying them at inference time to infer brain states or generate control information without re-deriving features or retraining representations for each task or user 122. This configuration enables the post-trained machine learning model 106 to perform inference using neural signals alone or in combination with available contextual information, while maintaining stable performance across variations in signal quality, recording conditions, or user behavior. In this manner, when the post-trained machine learning model 106 is being deployed in real-time, the output space of the model is constrained and no explicit task definitions, hand-engineered features, or per-session calibrations are needed, thereby enabling scalable, low-latency deployment across users, devices, and environments.

In some embodiments, the real-time neural signals 132 can be recorded via electrodes of a recording device 118 implanted within the user 122. For example, the real-time neural signals 132 can be recorded via electrodes 116 of a stent-electrode array 212 (see, e.g., FIG. 2B). The electrodes 116 can be implanted endovascularly, cortically, or subcortically within the brain of the user 122. As a more specific example, the real-time neural signals 132 can be recorded from within the brain of the user 122, locations along a surface of the brain of the user 122, locations exterior to brain vessels within the brain of the user 122, locations or spaces within a dura mater of the user 122, or a combination thereof.

In some embodiments, the real-time neural signals 132 can comprise at least one of neural signals that are temporally contiguous, temporally aligned, spatially aligned, and aligned by frequency. The neural signals recorded from the user 122 can comprise raw neural signals, transient oscillatory or pseudo-oscillatory bursts or burst features, binarized neural signals, action potentials, event-related potentials, graded potentials, local field potentials, rhythmic or repetitive patterns of neural signals, chunks of neural signals, or a combination thereof recorded across different recording channels, frequencies, and time.

In some embodiments, a pre-processing module running on the control unit 202 (see, e.g., FIG. 6) or the one or more servers 120 can filter the raw neural signals recorded from the recording device 118 in one or more desired frequency bands using one or more bandpass filters, wavelet convolutions, or a combination thereof. The pre-processing module can also convert voltage values of the filtered raw neural signals into power values (expressed as V²/Hz or μV²/Hz, dB/Hz, S² where S denotes the units of the signal, etc.) or normalized power values (expressed as z-scores, ratios, differences, percentage changes).

The pre-processing module can also apply at least one of a power threshold and a duration threshold for each of the desired frequency bands. The power threshold and/or the duration threshold can be selected or optimized for each user 122.

In some embodiments, the desired frequency bands can be between 0.1 Hz and 32 kHz. The desired frequency bands can also be between 4 Hz and 400 Hz. In certain embodiments, the desired frequency bands can be between 20 Hz and 200 Hz. In other embodiments, the desired frequency bands can be between 35 Hz and 150 Hz.

In some embodiments, the pre-processing module can apply at least one of a power threshold and a duration threshold to identify or detect a number of transient oscillatory or pseudo-oscillatory bursts from the neural signals recorded. For example, the pre-processing module can identify or detect the transient oscillatory or pseudo-oscillatory bursts in response to one of the magnitude or power-related values exceeding the power threshold and/or the duration threshold for each of the desired frequency bands.

The transient oscillatory or pseudo-oscillatory bursts can also be referred to as “transients,” “oscillation events,” “band-bursts (e.g., beta-bursts),” “band-events (e.g., gamma-events),” “miniature evoked responses,” or “oscillatory bursts.” The oscillatory or pseudo-oscillatory bursts can be characterized by being transient, meaning that each burst lasts for only a very short duration and that each burst is a high-energy burst, meaning that the power of each burst exceeds a threshold power level determined relative to a baseline level of neural activity and/or background noise.

In some embodiments, the duration of a typical transient oscillatory or pseudo-oscillatory burst can last between 1 ms to 100 ms. In other embodiments, the duration of a typical transient oscillatory or pseudo-oscillatory burst can last between 10 ms and 100 ms. The duration of a transient oscillatory or pseudo-oscillatory burst can depend on factors such as a frequency-band measured. For example, the transient oscillatory or pseudo-oscillatory burst can last between 1 ms to 10 ms when the frequency-band measured is relatively high (e.g., gamma-band) or last greater than 10 ms when the frequency-band measured is lower (e.g., alpha-band).

For example, the transient oscillatory or pseudo-oscillatory bursts can be referred to as beta bursts or beta-band bursts if these bursts were obtained from signals in the beta-oscillatory band (having a frequency of approximately 15-35 Hz). In addition, the transient oscillatory or pseudo-oscillatory bursts can be referred to as gamma bursts or gamma-band bursts if these bursts were obtained from signals in the gamma-oscillatory band (having a frequency of approximately 45-100 Hz). Moreover, the transient oscillatory or pseudo-oscillatory bursts can be referred to as alpha bursts or alpha-band bursts if these bursts were obtained from signals in the alpha-oscillatory band (having a frequency of approximately 7 Hz to 12 Hz). Furthermore, the transient oscillatory or pseudo-oscillatory bursts can be referred to as theta bursts or theta-band bursts if these bursts were obtained from signals in the theta-oscillatory band (having a frequency of approximately 4 Hz to 7 Hz).

The pre-processing module can extract one or more burst features from the transient oscillatory or pseudo-oscillatory bursts detected within a predetermined or preset detection period. The pre-processing module can detect upwards of hundreds of transient oscillatory or pseudo-oscillatory bursts within each detection period across the various electrodes 116 of the recording device 118 and across the various frequency bands (e.g., 0.1 Hz to 32 kHz).

In some embodiments, the detection period can be between 10 milliseconds (ms) and 100 ms. More specifically, the detection period can be between 50 ms and 100 ms. For example, the detection period can be about 100 ms.

The pre-processing module can extract the one or more burst features by counting or summing the number of transient oscillatory or pseudo-oscillatory bursts detected and determining the timing of such bursts. The pre-processing module can also determine the frequency, power value, and duration of each burst.

The burst features can comprise a burst count, a burst rate, a burst band frequency or frequency distribution, an interburst interval length (single channel and across multiple channels), a burst timing or timing pattern, an average burst duration, a burst waveform (e.g., the time domain waveform of a burst), or any changes or combination thereof. The burst features can also comprise an average power across bursts within a window of time, a maximum power of the bursts, a number of cycles, a peak frequency of the bursts, a minimum frequency of the bursts, a maximum frequency of the bursts, a frequency span (expressed in octaves), an average power just before and/or just after a burst, a low-frequency instantaneous phase at the time of a high-frequency burst, alpha and beta power at the time of a high-frequency burst, an oscillatory score (i.e., a correlation between the filtered and raw signal at the time of a burst). The burst features can also comprise a burst synchronization or distance (i.e., a measure of the correlation between bursts at different channels when treated as a point process), the left and/or right slope of the transient bursts (i.e., how fast does the amplitude rise or fall), and repeating sequences in time of transient bursts (e.g., certain user thoughts or movement types can generate a sequence of bursts that appear at certain electrodes at specific time intervals).

In other embodiments, the recording device 118 can be a non-invasive recording device. In these embodiments, the real-time neural signals 132 can be recorded via electrodes 310 of an EEG device 308 such as an EEG cap (see, e.g., FIG. 3C) worn by the user 122.

In some embodiments, the user 122 can use the post-trained machine learning model 106 to control the one or more devices 108 or software applications 110 running on such devices 108 without requiring any real-time contextual information 136 being inputted into the post-trained machine learning model 106. That is, inputting the real-time neural signals 132 into the post-trained machine learning model 106 can be undertaken without any real-time contextual information 136 being inputted into the post-trained machine learning model 106 along with the real-time neural signals 132 to generate the control information 134. In these embodiments, only real-time neural signals 132 are provided as inputs to the post-trained machine learning model 106 to generate the control information 134.

In other embodiments, real-time contextual information 136 can be provided along with the real-time neural signals 132. In these embodiments, inputting the real-time neural signals 132 into the post-trained machine learning model 106 can be undertaken with real-time contextual information 136 also being inputted into the post-trained machine learning model 106 to generate the control information 134. In certain embodiments, the real-time contextual information 136 can be temporally aligned with the real-time neural signals 132 before being input into the post-trained machine learning model 106.

The real-time contextual information 136 can be obtained from a wearable display device 208 (see, e.g., 8A and 8B) worn by the user 122 or another type of portable device 126 (e.g., a smartphone, a tablet computer, laptop computer, a digital video camera, an audio recorder, etc.) carried by the user 122 or within a vicinity of the user 122, a device or server controlling the wearable display device 208 or another type of portable device 126, or another a computing device, tablet, smartphone, or server communicatively coupled to the wearable display device 208 or another type of portable device 126.

The real-time contextual information 136 can refer to data or information concerning a time-of-day, a day-of-the-week, a month, a year, a season, a setting or location, a weather condition, a detected activity, one or more objects or individuals detected within a real environment surrounding the user 122 or a simulated environment viewed or otherwise experienced by the user 122, one or more connected devices detected, or a combination thereof.

The real-time contextual information 136 can be in the form of text labels or text-based files, semantic labels, tags, keywords, metadata, numerical labels, key-value pairs, machine-readable data structures, sensor data or sensor logs, device data or device logs, audio data or audio file(s), image data or an image file(s), and/or video data or video file(s). The real-time contextual information 136 can be data or information outputted by one or more object-detection machine learning models.

The post-trained machine learning model 106 can be used by different users 122 without having to re-train the model. In some embodiments, the post-trained machine learning model 106 can be further fine-tuned for each individual user 122. The post-trained machine learning model 106 can also be used to accomplish different tasks without having to re-train the model.

In some embodiments, the control information 134 can be transmitted by one or more servers 120 running the post-trained machine learning model 106. In other embodiments, the control information 134 can be transmitted by one or more servers 120 to a control unit 202 (see, e.g., FIG. 6) or another computing device within a vicinity of the user 122 to then transmit to the one or more devices 108 via a wireless communication protocol (e.g., Bluetooth™ or WiFi).

In some embodiments, the control information 134 can be generated based on brain-state embeddings that align neural and contextual representations. The method 100C can also comprise controlling the one or more devices 108 or the one or more software applications 110 running on such devices 108 based on the control information 134. The real-time neural signals 132 recorded can be provided as inputs to the post-trained machine learning model 106 and the incoming real-time neural signals 132 can be processed by the post-trained machine learning model 106 using representations learned during the pre-training phase and adapted during the post-training phase. In some embodiments, the post-trained machine learning model 106 can encode the real-time neural signals 132 into an inferred brain state or a behavioral representation within the model's latent space. This inferred brain state or brain-state embedding can be an internal representation reflecting the current neural condition of the user 122 and can be produced implicitly as part of the model's forward computation rather than as an explicitly labeled output. Based on the inferred brain-state representation, the post-trained machine learning model 106 can output control information 134 that is suitable for controlling one or more devices 108 or software applications running on such devices 108.

In some embodiments, the one or more devices 108 can comprise a personal computing device 140, a smart-home device 142 or Internet-of-Things (IoT) device, a robotic device 144 or robotic component, a mobility vehicle 146, or a combination thereof. For example, the personal computing device 140 can comprise a laptop computer, a desktop computer, a smartphone, a tablet computer, or a combination thereof. Also, for example, the smart-home device 142 or IoT device can comprise a smart lamp (e.g., a Bluetooth™-enabled or WiFi-enabled lamp), a smart fan (e.g., a Bluetooth™-enabled or WiFi-enabled fan), a smart pet feeder (e.g., a Bluetooth™-enabled or WiFi-enabled pet feeder), a smart refrigerator (e.g., a Bluetooth™-enabled or WiFi-enabled refrigerator), a smart washer or dryer (e.g., a Bluetooth™-enabled or WiFi-enabled washer or dryer), or a smart cooking appliance (e.g., a Bluetooth™-enabled or WiFi-enabled cooking appliance). As another example, the robotic device 144 or the robotic component can comprise a robotic arm, a robotic hand, robotic legs, a robotic exoskeleton, or a general-purpose humanoid robot. As an additional example, the mobility vehicle 146 can comprise an electric wheelchair or an electric mobility scooter.

In some embodiments, controlling the one or more devices 108 based on the control information 134 can further comprise transmitting device control signals that cause the one or more devices 108 to perform or cease from performing a physical or operational action. In certain embodiments, the brain-state embeddings can modulate, gate, delay, or parameterize the control information 134.

For example, when the device 108 to be controlled is a personal computing device 140, a device control signal or interrupt command can be transmitted to one or more processors (e.g., a CPU) of the personal computing device 140 to initiate a keyboard press or stop a keyboard press. Also, for example, when the device 108 is a motorized or electric wheelchair, a device control signal can be transmitted to a control unit of the motorized or electric wheelchair to steer or drive the wheelchair or stop the wheelchair.

In some embodiments, the control information 134 can be one or more output signals representing inferred brain-state-conditioned control information usable by a downstream system. The control information 134 can comprise digital motor outputs or outputs that condition, modulate, gate delay, parameterize, suppress execution of, prioritize, and/or multiplex actions or signals. The control information 134 can control, modify, inhibit, delay, prioritize, or otherwise condition operation of one or more downstream devices, systems, and/or software applications. The control information 134 can refer to motor-related or non-motor-related controls or signals. The control information 134 can also indirectly modulate or inhibit control signals or actions.

For example, the control information 134 can cause the presentation of an action list UI. Also, for example, the control information 134 can pause, inhibit, or condition execution of a control signal or digital motor output.

In some embodiments, the post-trained machine learning model 106 can comprise one or more classifiers, one or more decoder heads, or a combination thereof. The one or more classifiers and/or the one or more decoder heads can assist the post-trained machine learning model 106 in selecting the appropriate control information 134.

As used herein, control information 134 can refer broadly to one or more output signals generated by the post-trained machine learning model 106 or output signals encoding information derived from inferred brain states or brain-state embeddings. Control information 134 can represent brain-state-conditioned information usable by one or more downstream systems, devices, software applications, or control modules to influence operation, execution, or behavior.

Control information 134 can be generated based solely on real-time neural signals 132 recorded from the user 122, or can be generated based on a combination of real-time neural signals 132 and real-time contextual information 136, such as contextual information obtained from wearable display devices 208, environmental sensors, system logs, object-detection models, or other contextual data sources described herein (see, e.g., FIGS. 6-8B). In such embodiments, the control information 134 can reflect both the inferred brain state of the user 122 and contextual conditions present during real time use. Control information 134 is not limited to direct actuation commands and does not require direct physical or virtual motion to occur.

In some embodiments, the control information 134 can comprise digital motor outputs (DMOs) that directly cause physical or virtual actuation, such as cursor movement, selection events, robotic actuation, mobility control, or manipulation of physical or virtual objects (see, e.g., FIGS. 1C, 5A-5C, 6, 7A-7B, and 8A-8B). In other embodiments, the control information 134 can comprise non-motor digital outputs, including output signals that do not directly actuate any physical or virtual motion. Non-motor digital outputs can include output signals configured to condition operation of a downstream system, rather than commanding a specific action. Such conditioning can include, without limitation: gating or inhibiting execution of an action; delaying or deferring execution of an action; suppressing execution of a control signal; parameterizing or modulating downstream control behavior; prioritizing or arbitrating among multiple candidate actions; and multiplexing control signals across devices or subsystems.

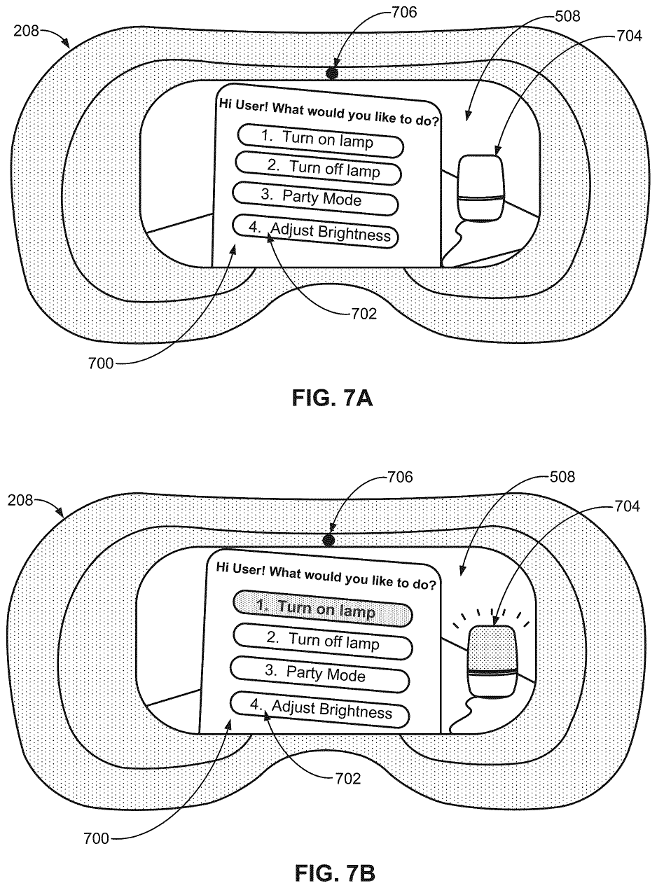

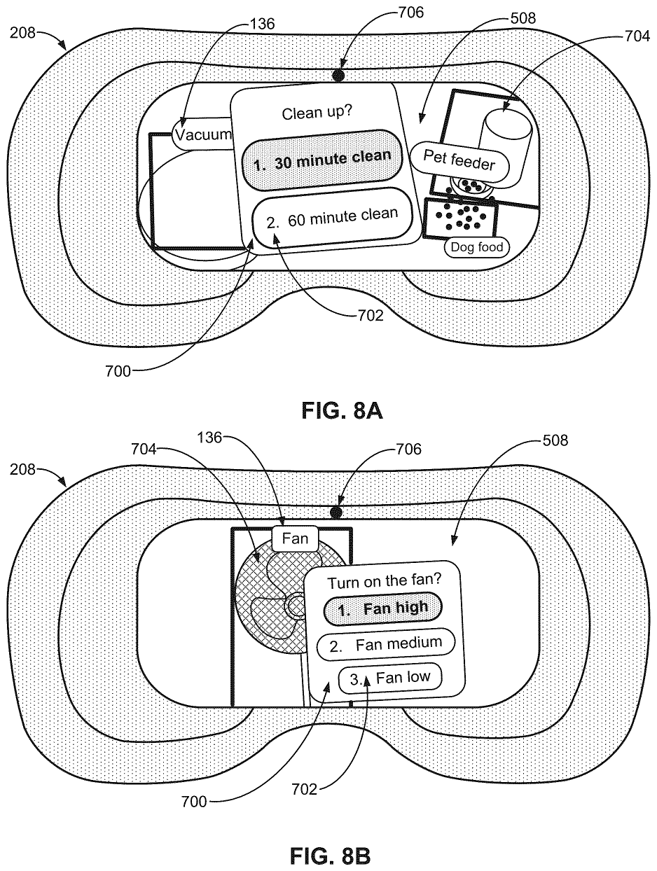

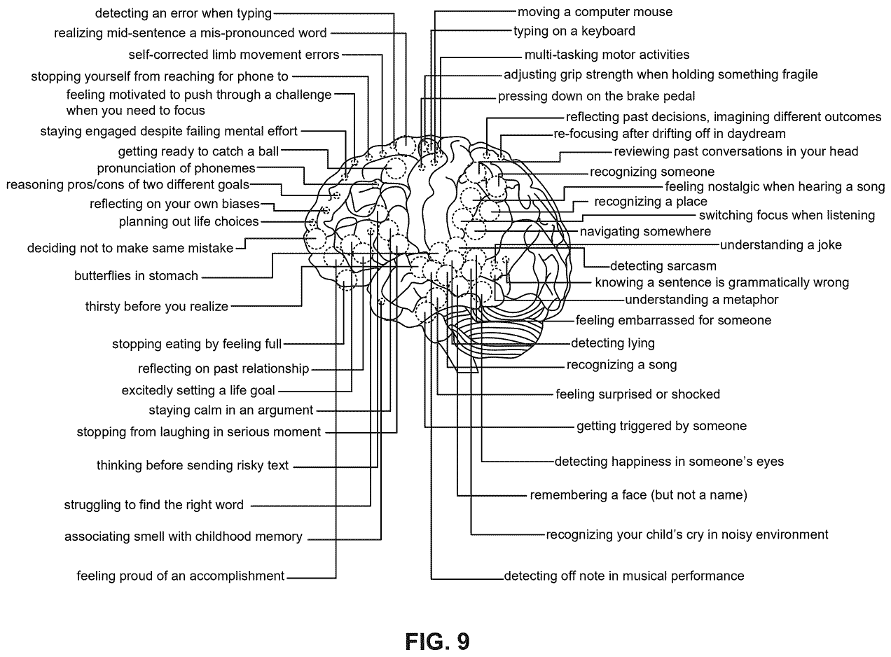

For example, as described in connection with FIGS. 7A-8B, control information 134 can cause generation or modification of an action list user interface (e.g., an action list UI 700), ordering or filtering candidate actions based on inferred brain states and real-time contextual information. In these embodiments, the control information 134 can influence which actions are presented, how they are presented, or whether an action is executed, without directly actuating a device at the time the control information is generated. In additional embodiments, the control information 134 can include user interface modulation, such as causing presentation, highlighting, suppression, confirmation prompts, warnings, reminders, or informational displays. For example, the control information 134 can pause message transmission, require confirmation before executing an irreversible action, highlight text for correction, or surface contextual information without initiating an action (see, e.g., examples described in connection with FIG. 9). In certain embodiments, the control information 134 can comprise intermediate or indirect output representations consumed by downstream logic, agents, controllers, or mapping modules. Such output representations can influence system behavior by controlling how, when, whether, or under what parameters a downstream action is performed, even when no direct actuation occurs. Accordingly, digital motor outputs represent one non-limiting subset of control information 134, and the control information 134 can include output signals or representations usable to control, modify, inhibit, delay, prioritize, arbitrate, parameterize, or otherwise condition system behavior, whether or not such output signals directly cause physical or virtual actuation.

As shown in FIG. 1C, in some embodiments, the post-trained machine learning model 106 can be run on one or more servers 120 in a cloud computing environment 121 (i.e., in the cloud). As will be discussed in more detail in the following sections, the recording device 118 can be connected to a telemetry unit 204 that is communicatively coupled (e.g., via wireless or wired connections) to a control unit 202 (see, e.g., FIG. 6).

The control unit 202 can also be communicatively coupled to the one or more servers 120. The control unit 202 can transmit the real-time neural signals 132 to the one or more servers 120 in order to input the real-time neural signals 132 to the post-trained machine learning model 106.

In some embodiments, the control unit 202 can also be communicatively coupled to a wearable display device 208 worn by the user 122 or another type of portable device 126 carried by the user 122 or within a vicinity of the user 122, or a device or server controlling the wearable display device 208 or the portable device 126. In these embodiments, at least some of the real-time contextual information 136 recorded by or otherwise obtained from the wearable display device 208 and/or another type of portable device 126 can be transmitted from the control unit 202 to the one or more servers 120.

In other embodiments, the one or more servers 120 can retrieve or otherwise obtain the real-time contextual information 136 directly from the wearable display device 208 worn by the user 122 or another type of portable device 126 carried by the user 122 or within a vicinity of the user 122.

FIG. 2A illustrates one embodiment of a system 200 that can used to train (e.g., pre-train and/or post-train) a neural foundation model (see, e.g., FIGS. 1A-1C). The system 200, or parts thereof, can also be used to control one or more devices 108 (see, e.g., FIG. 1C) or one or more software applications 110 running on such devices 108 based on predictions outputted by the neural foundation model. The system 200 can also be used to undertake any of the methods 100A, 100B, or 100C disclosed herein (see, e.g., FIGS. 1A-1C).

The system 200 can comprise the recording device 118 (see FIG. 2B), a control unit 202, a telemetry unit 204, and one or more communication conduits 206 (e.g., lead wires) connecting the telemetry unit 204 to the recording device 118. In some embodiments, the system 200 can further comprise a wearable display device 208 and/or a screen display 210.

In some embodiments, the wearable display device 208 can be a virtual-reality (VR) headset 502 (see, e.g., FIG. 5A). In other embodiments, the wearable display device 208 can be an augmented-reality (AR) wearable 510 (see, e.g., FIG. 5B). In additional embodiments, the wearable display device 208 can be a mixed-reality headset. For example, the wearable display device 208 can be the Apple Vision Pro® headset.

In alternative embodiments, the wearable display device 208 can be a pair of smart glasses or other type of smart headwear.

The recording device 118 can be configured to record the neural activity of a subject 114 or a user 122 in the form of neural signals. In some embodiments, the recording device 118 can be an invasive recording device 118 configured to be implanted within a brain of the subject 114 or the user 122. For example, the recording device 118 can be a stent-electrode array 212 configured to be implanted within a brain vessel 214 of the subject 114 or the user 122 (see, e.g., FIG. 2B). As a more specific example, the recording device 118 can be implanted within a cortical or cerebral vein or sinus of the subject 114 or the user 122.

For example, the recording device 118 can be implanted within a superior sagittal sinus, an inferior sagittal sinus, a sigmoid sinus, a transverse sinus, a straight sinus, a superficial cerebral vein such as a vein of Labbe, a vein of Trolard, a Sylvian vein, a Rolandic vein, a deep cerebral vein such as a vein of Rosenthal, a vein of Galen, a superior thalamostriate vein, an inferior thalamostriate vein, or an internal cerebral vein, a central sulcal vein, a post-central sulcal vein, or a pre-central sulcal vein. In certain embodiments, the recording device 118 can be implanted within a vessel extending through the hippocampus or amygdala of the user or subject.

FIG. 2B illustrates that the stent-electrode array 212 can comprise a plurality of electrodes 116 affixed, secured, or otherwise coupled to an exterior portion or radially outer portion of an expandable stent 216 or scaffold serving as an endovascular carrier for the electrode array. For example, the electrodes 116 can be arranged along filaments making up the walls, rings, or scaffold of the expandable stent 216.

In some embodiments, the recording device 118 can comprise typically between 8 to 24 electrodes. For example, the recording device 118 can comprise 16 electrodes. In other embodiments, the recording device 118 can comprise between 24 and 64 electrodes 116.

In some embodiments, the filaments of the expandable stent 216 can be made in part of a shape-memory alloy. For example, the filaments of the expandable stent 216 can be made in part of Nitinol or Nitinol wire. The filaments of the expandable stent 216 can also be made in part of stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof. In alternative embodiments, the filaments of the expandable stent 216 can also be made in part of a shape memory polymer.

The electrodes 116 can be made in part of platinum, platinum black, gold, iridium, palladium, rhodium, or alloys or composites thereof (e.g., a gold-palladium-rhodium alloy or composite). In certain embodiments, the electrodes 116 can be made of a metal alloy or composite with a high charge injection capacity (e.g., a platinum-iridium alloy or composite).

The electrodes 116 can be shaped as circular disks having a disk diameter of between about 100 μm to 1.0 mm. In other embodiments, the electrodes 116 can have a disk diameter of between 1.0 mm and 1.5 mm. In other embodiments, the electrodes 116 can be cylindrical, spherical, cuff-shaped, ring-shaped, partially ring-shaped (e.g., C-shaped), or semi-cylindrical.

In other embodiments, the stent-electrode array 212 can be any of the stents, scaffolds, stent-electrodes, or stent-electrode arrays disclosed in U.S. Patent Pub. No. 2025/0041592; U.S. Patent Pub. No. 2021/0365117; U.S. Patent Pub. No. 2021/0361950; U.S. Patent Pub. No. 2020/0363869; U.S. Patent Pub. No. 2020/0078195; U.S. Patent Pub. No. 2020/0016396; U.S. Patent Pub. No. 2019/0336748; U.S. Patent Pub. No. US 2014/0288667; U.S. Pat. Nos. 10,575,783; 10,485,968; 10,729,530; and 10,512,555; the contents of which are incorporated herein by reference in their entireties.

When the recording device 118 (e.g., the stent-electrode array 212) is implanted within a brain vessel 214 of the user or subject, each of the electrodes 116 of the recording device 118 can be configured to read or record the electrical activities of neurons within a vicinity of each electrode 116. The electrical activities of neurons can be recorded as raw electrical signals. As will be discussed in more detail in later sections, the raw electrical signals can be filtered and processed to detect one or more transient oscillatory or pseudo-oscillatory bursts.

The raw electrical signals can be divided into bands by their frequency. For example, the desired frequency bands comprise frequency bands between 0.1 Hz and 32 kHz.

In other embodiments, the recording device 118 can be an implantable microelectrode array (MEA). For example, the recording device 118 can be a Utah microelectrode array or a Michigan microelectrode array. In certain embodiments, the recording device 118 can be a thin-film electrode array or comprised of thin-film microelectrodes.

As a more specific example, the microelectrode array can have an array portion comprising at least about 100 electrodes, 200 electrodes, 256 electrodes, 512 electrodes, 1024 electrodes, or more. The plurality of electrodes can be positioned in M rows and N columns across the array portion. The array portion can comprise about or at least about 5, 10, 16, 32, 64 columns, or any range of values therebetween. The array portion can comprise about or at least about 5, 10, 16, 32, 64 rows, or any range of values therebetween. The electrodes can be spaced apart from one another at a pitch of about or at least about 0.1 mm, 0.25 mm, 0.5 mm, 1 mm, 1.5 mm, 2 mm, 2.5 mm, 5 mm, 10 mm, 20 mm, 30 mm, or any range of values therebetween. The electrodes can be AC-coupled single-ended inputs which can be referenced to a selectable reference node. Each electrode can have an impedance of less than 50 kΩ at 1 kHz.

In further embodiments, the recording device 118 can be an electrode array that can be implanted on a brain surface or a surface of the cortex. For example, the recording device 118 can be an electrocorticography (eCoG) electrode array (see, e.g., FIG. 3D).

In other embodiments, the recording device 118 can be a non-invasive recording device 118. For the example, the recording device 118 can be an EEG device or EEG cap (see, e.g., FIG. 3C).

FIG. 2C illustrates that one or more communication conduits 206 (e.g., lead wires) can connect the recording device 118 (e.g., the stent-electrode array 212) with the telemetry unit 204 communicatively coupled (e.g., via wireless or wired connections) to the control unit 202. Alternatively, the recording device 118 can be communicatively coupled directly with the control unit 202.

The communication conduits 206 can be biocompatible lead wires or cables. When the recording device 118 is a stent-electrode array 212 deployed within a brain vessel 214 of the subject 114 or the user 122, the communication conduits 206 can extend through one or more brain vessels and out through a wall of a vein connected to a major vein (e.g., the internal jugular vein) of the subject 114 or the user 122. The communication conduits 206 can then tunnel under the skin of the subject 114 or the user 122 to a body part of the subject 114 or the user 122 where the telemetry unit 204 is implanted (e.g., beneath the pectoralis major muscle).

FIG. 2D illustrates a close-up view of an embodiment of the telemetry unit 204. In some embodiments, the telemetry unit 204 can be configured to transmit signals received from the recording device 118 to the control unit 202 for processing and analysis. The telemetry unit 204 can also serve as a communication hub between the recording device 118 and the control unit 202.

In certain embodiments, the telemetry unit 204 can be an internal telemetry unit 204 implantable under the skin of the subject 114 or the user 122. For example, the telemetry unit 204 can be implanted within the body of the subject 114 or the user 122. As a more specific example, the telemetry unit 204 can be implanted within a pectoral region, a subclavian space, or an arm or forearm of the subject 114 or the user 122.

In other embodiments, the telemetry unit 204 can be an external telemetry unit 204 not implanted within the subject 114 or the user 122. In these embodiments, the communication conduit 206 can extend through the skin of the user or subject to connect to the telemetry unit 204. In additional embodiments, the telemetry unit 204 can comprise both an implantable portion and an external portion.

In some embodiments, the telemetry unit 204 can transmit data or signals to the control unit 202 or an edge device and receive data or commands from the control unit 202 or the edge device via a wired connection. In other embodiments, the telemetry unit 204 can transmit data or signals to the control unit 202 or the edge device or receive data or commands from the control unit 202 or the edge device via a wireless communication protocol such as Bluetooth™, Bluetooth Low Energy (BLE), ZigBee™, WiFi, or a combination thereof.

The control unit 202 or the edge device can also be communicatively coupled to the wearable display device 208 and/or the screen display 210. Alternatively, the control unit 202 can be communicatively coupled to another device that is used to control the wearable display device 208 and/or the screen display 210. The control unit 202 or the edge device can receive data or information from the wearable display device 208, the screen display 210, or the device controlling the wearable display device 208 or the screen display 210 concerning what is currently being rendered or displayed via the wearable display device 208 or the screen display 210 in real-time or near real-time. The control unit 202 or the edge device can also receive data or information from the wearable display device 208 and/or the screen display 210 or the device controlling the wearable display device 208 or the screen display 210 concerning what was previously rendered or displayed via the wearable display device 208 or the screen display 210.

The control unit 202 can refer to a customized computing device configured to interact with the telemetry unit 204 and/or the recording device 118. In other embodiments, the control unit 202 can refer to a personal computing device such as a desktop computer, a laptop computer, or a tablet computer.

The control unit 202 can comprise one or more processors, memory and storage units, and wireless communication modules. The processors can include one or more CPUs, GPUs, ASICs, FPGAs, or a combination thereof. The processors can execute software stored in the memory and storage units to execute the methods or instructions described herein.

The memory and storage units can comprise volatile memory and non-volatile memory or storage. For example, the memory and storage units can comprise flash memory or storage such as one or more solid-state drives, dynamic random access memory (DRAM) or synchronous dynamic random access memory (SDRAM) such as low-power double data rate (LPDDR) SDRAM and embedded multi-media controller (eMMC) storage. The memory and storage units can store software instructions, firmware, data, tables, logs, databases, or a combination thereof.

The wireless communication modules can comprise at least one of a cellular communication module, a WiFi communication module, a Bluetooth® communication module, or a combination thereof. For example, the cellular communication module can support communications over a 5G network or a 4G network (e.g., a 4G long-term evolution (LTE) network) with automatic fallback to 3G networks. The cellular communication module can comprise a number of embedded SIM cards or embedded universal integrated circuit cards.

The control unit 202 can communicate with one or more servers 120 (see, e.g., FIGS. 1A-1C) over one or more networks. In some embodiments, the one or more networks can refer to one or more wide area networks (WANs) such as the Internet or other smaller WANs, wireless local area networks (WLANs), local area networks (LANs), wireless personal area networks (WPANs), system-area networks (SANs), metropolitan area networks (MANs), campus area networks (CANs), enterprise private networks (EPNs), virtual private networks (VPNs), multi-hop networks, or a combination thereof. The one or more servers 120 and the control unit 202 can connect to the one or more networks using any number of wired connections (e.g., Ethernet, fiber optic cables, etc.), wireless connections established using a wireless communication protocol or standard such as a 3G wireless communication standard, a 4G wireless communication standard, a 5G wireless communication standard, a long-term evolution (LTE) wireless communication standard, a Bluetooth™ (IEEE 802.15.1) or Bluetooth™ Lower Energy (BLE) short-range communication protocol, a wireless fidelity (WiFi) (IEEE 802.11) communication protocol, an ultra-wideband (UWB) (IEEE 802.15.3) communication protocol, a ZigBee™ (IEEE 802.15.4) communication protocol, or a combination thereof.

The control unit 202 can transmit data and files to the one or more servers 120 and receive data and files from the one or more servers 120 via secure connections. The secure connections can be real-time bidirectional connections secured using one or more encryption protocols such as a secure sockets layer (SSL) protocol, a transport layer security (TLS) protocol, or a combination thereof. Additionally, data or packets transmitted over the secure connection can be encrypted using a Secure Hash Algorithm (SHA) or another suitable encryption algorithm. Data or packets transmitted over the secure connection can also be encrypted using an Advanced Encryption Standard (AES) cipher.

Software instructions run on the control unit 202 can be written in the Objective-C programming language, Swift® programming language, Java® programming language, JavaScript programming language, Python® programming language, C++programming language, or a combination thereof.

As previously discussed, at least part of the system 200 can be used to pre-train and then post-train a neural foundation model (see, e.g., FIGS. 1A-1C). The post-trained neural foundation model 106 can later be used to control one or more devices 108 or a software application (e.g., a software application running on such devices 108).

FIG. 3A illustrates another embodiment of the implantable recording device 118 as a coiled wire 300 comprising a plurality of electrodes 301. The coiled wire 300 can serve as the endovascular carrier for the electrodes 301 and can be used in vessels that are too small to accommodate the stent-electrode array.

In some embodiments, the electrodes 301 can be made of the same material as the electrodes 116. The electrodes 301 can be adapted to fit along the length of the coiled wire 300.

The coiled wire 300 can be a biocompatible wire or microwire configured to wind itself into a coiled pattern or a substantially helical pattern. The electrodes 301 can be arranged such that the electrodes 301 are scattered along a length of the coiled wire 300. More specifically, the electrodes 301 can be affixed, secured, or otherwise coupled to distinct points along a length of the coiled wire 300.

The electrodes 301 can be separated from one another such that no two electrodes 301 are within a predetermined separation distance (e.g., at least 10 μm, at least 100 μm, or at least 1.0 mm) from one another. In some embodiments, the coiled wire 300 can carry between 8 to 24 electrodes. For example, the coiled wire 300 can carry 16 electrodes. In other embodiments, the coiled wire 300 can carry between 24 and 64 electrodes 301.

In some embodiments, the wire 300 can be configured to automatically wind itself into a coiled configuration (e.g., helical pattern) when the wire 300 is deployed out of a delivery catheter. For example, the coiled wire 300 can automatically attain its coiled configuration via shape memory when the delivery catheter or sheath is retracted. The coiled configuration or shape can be a preset or shape memory shape of the wire 300 prior to the wire 300 being introduced into a delivery catheter. The preset or pre-trained shape can be made to be larger than the diameter of the anticipated deployment or implantation vessel to enable the radial force exerted by the coils to secure or position the coiled wire 300 in place within the deployment or implantation vessel.

The wire 300 can be made in part of a shape-memory alloy, a shape-memory polymer, or a combination thereof. For example, wire 300 can be made in part of Nitinol (e.g., Nitinol wire). The wire 300 can also be made in part of stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof.

FIG. 3B illustrates yet another embodiment of the implantable recording device 118 as an anchored wire 302 comprising a plurality of electrodes 301. The anchored wire 302 can serve as the endovascular carrier for the electrodes 301 and can be used in vessels that are too small to accommodate either the coiled wire 300 or the stent-electrode array 212.

The anchored wire 302 can comprise a biocompatible wire or microwire attached or otherwise coupled to an anchor or another type of endovascular securement mechanism. FIG. 3B illustrates that the anchored wire 302 can comprise a barbed anchor 304, a radially-expandable anchor 306, or a combination thereof (both the barbed anchor 304 and the radially-expandable anchor 306 are shown in broken or phantom lines in FIG. 3B). In some embodiments, the barbed anchor 304 can be positioned at a distal end of the anchored wire 302. In other embodiments, the barbed anchor 304 can be positioned along one or more sides of the wire or microwire. The barbs of the barbed anchor 304 can secure or moor the anchored wire 302 to an implantation site within the user or subject. The radially-expandable anchor 306 can be a segment of the wire or microwire shaped as a coil or loop. The coil or loop can be sized to allow the coil or loop to conform to a vessel lumen and to expand against a lumen wall to secure the anchored wire 302 to an implantation site within the vessel. For example, the coil or loop can be sized to be larger than the diameter of the anticipated deployment or implantation vessel to enable the radial force exerted by the coil or loop to secure or position the anchored wire 302 in place within the deployment or implantation vessel.

The electrodes 301 of the anchored wire 302 can be scattered along a length of the anchored wire 302. More specifically, the electrodes 301 can be affixed, secured, or otherwise coupled to distinct points along a length of the anchored wire 302. The electrodes 301 can be separated from one another such that no two electrodes 301 are within a predetermined separation distance (e.g., at least 10 μm, at least 100 μm, or at least 1.0 mm) from one another.

In some embodiments, the anchored wire 302 can carry between 8 to 24 electrodes. For example, the anchored wire 302 can carry 16 electrodes. In other embodiments, the anchored wire 302 can carry between 24 and 64 electrodes 301.

Although FIG. 3B illustrates the anchored wire 302 having only one barbed anchor 304 and one radially-expandable anchor 306, it is contemplated by this disclosure that the anchored wire 302 can comprise a plurality of barbed anchors 304 and/or radially-expandable anchors 306.

FIG. 3C illustrates one embodiment of an electroencephalogram (EEG) device 308 or EEG cap serving as the recording device 118. The EEG device 308 can be a non-invasive head-mounted EEG apparatus or headgear. For example, the EEG device 308 can be an EEG cap or an EEG-visor configured to be worn by a subject or user. The EEG device 308 can comprise a plurality of non-invasive electrodes 310 configured to be in contact with the scalp of the subject 114 or user 122.

In some embodiments, the brain activity or neural signals detected by the EEG device 308 can be neural oscillations or brainwaves of the subject 114 or user 122, similar to those recorded by the implantable recording device 118. For example, the EEG device 308 can record neural oscillations, including any changes in such neural oscillations, over time in the beta-band (about 14 Hz to 30 Hz), alpha frequency range or alpha-band (about 7 Hz to 12 Hz), theta frequency range or theta-band (about 4 Hz to 7 Hz), gamma frequency range or gamma-band including a low frequency gamma-band (about 30 Hz to 70 Hz) and a high frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The EEG device 308 can record changes in the power of such neural oscillations (e.g., as measured in decibels (dBs), micro-volts squared per Hz (μV2/Hz), average t-scores, average z-scores, etc.).

FIG. 3D illustrates one embodiment of an electrocorticography (ECoG) device 312 serving as the recording device 118. The ECoG device 312 (also referred to as an intracranial EEG device) can be a flexible or stretchable electrode-mesh or one or more electrode patches or electrodes implanted or placed on a surface of the brain of the subject 114 or user 122. The electrode-mesh or electrode patch can comprise a plurality of electrodes 314 arranged on the mesh or patch, respectively.

The brain activity detected by the ECoG device 312 can be neural oscillations or brainwaves of the subject 114 or user 122, similar to those recorded by the stent-electrode array 212. For example, the ECoG device 312 can record neural oscillations, including any changes in such neural oscillations, over time in the beta-band (about 14 Hz to 30 Hz), alpha frequency range or alpha-band (about 7 Hz to 12 Hz), theta frequency range or theta-band (about 4 Hz to 7 Hz), gamma frequency range or gamma-band including a low frequency gamma-band (about 30 Hz to 70 Hz) and a high frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The ECoG device 312 can record changes in the power of such neural oscillations (e.g., as measured in decibels (dBs), micro-volts squared per Hz (μV2/Hz), average t-scores, average z-scores, etc.).

FIG. 3E illustrates one embodiment of a functional magnetic resonance imaging (fMRI) device 316 serving as the recording device 118. The fMRI device 316 can detect changes in blood flow and blood-oxygen levels within the brain.

In some embodiments, the fMRI device 316 can measure brain activity using blood-oxygen-level dependent (BOLD) contrast imaging. For example, brain activity can be expressed as changes in the BOLD signal. In other embodiments, the fMRI device 316 can measure brain activity using arterial spin labeling (ASL) rather than BOLD contrast imaging.

FIG. 3F illustrates one embodiment of a functional near infrared spectroscopy (fNIRS) device 318 serving as the recording device 118. The fNIRS device 318 can use near-infrared light (NIR) to measure hemodynamic activity in the brain of a subject or user. For example, the fNIRS device 318 can comprise a fNIRS cap configured to worn on the head of the subject or user. The fNIRS device 318 can comprise a plurality of NIR light sources and detectors (called optodes). The fNIRS device 318 can measure the hemodynamic activity by measuring changes in oxy-hemoglobin concentrations (HbO) and deoxy-hemoglobin (HbR) concentrations in the cerebral cortex.

FIG. 4A illustrates one example implementation of a method of pre-training and post-training a machine learning model 400 using raw neural signals 402 discretized as neural tokens 404. In this embodiment, the machine learning model 400 can comprise a burst transformer 406 comprising a transformer backbone 408 and a detector 410 comprising a decoder head 412.

Raw neural signals 402 can be recorded or otherwise acquired from the brain of a subject via one or more electrodes 116 of a recording device 118. These raw neural signals 402 can comprise time-varying electrical activity reflecting spatiotemporal neural dynamics. The raw neural signals 402 can be provided to a neural tokenizer module 414, which transforms the continuous raw neural signals 402 into discrete neural tokens 404. The neural tokenizer module 414 can be an example of a pre-processing module.

As shown in FIG. 4A, the neural tokenizer module 414 can apply one or more frequency-domain transformations, such as high-frequency filter banks 416, to the raw neural signals 402. The filtered signals are then processed to detect oscillatory burst events. Each detected oscillatory burst is encoded as a discrete neural token 404. The resulting neural tokens 404 are arranged into ordered sequences 418 representing temporal neural activity. By way of example, approximately forty-seven hours of raw neural data can be converted into approximately seventeen million neural tokens 404.

In some embodiments, each chunk of raw neural signals 402 can be a 10 millisecond (ms) recording. Each chunk of raw neural signals 402 (e.g., a 10 ms recording) can be turned into a discrete neural token 404. This means that a neural signal recording lasting only a few seconds (e.g., 3 seconds to 5 seconds) can yield thousands of neural tokens 404 across the various electrodes 116 of the recording device 118 and across the various desired frequency bands. Also, for example, a raw neural recording lasting approximately five minutes can be converted into tens of thousands of neural tokens 404. These token sequences 418 can then be provided as inputs to a machine learning model comprising a transformer-based architecture. The machine learning model 400 can be pre-trained to predict one or more future neural tokens conditioned on a preceding neural token sequence 418. Pre-training can be performed using a curriculum-based autoregressive objective, in which the prediction horizon and the sequence complexity are progressively increased over training iterations.

The pre-trained machine learning model can comprise approximately five hundred fifty-eight thousand trainable parameters, although other model sizes and architectures can be used. The pre-training process produces latent embeddings that encode neural dynamics associated with cognitive processes, intention, and internal brain states, without requiring explicit behavioral labels. As a result, neural latent representations can be learned that are closer to the source of cognition than machine learning models trained solely on externally observable outputs, such as language or motor actions, and provides a foundation for efficient downstream adaptation to task-specific decoding and control applications.

FIG. 4A also illustrates that channel embeddings 420 and positional encodings 422 can be combined with the neural token sequences 418 and processed by a transformer backbone 408 of the burst transformer 406. During this pre-training phase, the transformer backbone 408 is trained to predict future neural tokens using a curriculum-based autoregressive training process.

FIG. 4A also illustrates that post-training of the machine learning model 400 can be performed by training a decoder head 412 of the detector 410 while maintaining the pre-trained transformer backbone 408 of the machine learning model 400. Besides the decoder head, the detector 410 can also comprise one or more convolutional layers, pooling operations, and temporal weighting operations. These components of the detector 410 can be configured to aggregate temporal information from the pretrained embeddings and generate a classification output indicating whether a given neural event corresponds to an intended or unintended action.

The decoder head 412 can be trained to map the pretrained embeddings to task-specific outputs. The decoder head 412 can classify neural activity corresponding to intended versus unintended brain-derived control signals. The decoder head 412 can comprise substantially fewer parameters than the pretrained transformer backbone 408, enabling efficient adaptation with limited labeled data. The resulting machine learning model 400 is capable of robust real-world inference by distinguishing intentional neural commands from background or unintended neural activity, without requiring full retraining of the model.

FIG. 4B (top image) illustrates true oscillatory burst patterns extracted from neural data as well as the corresponding predicted burst probabilities (bottom image) outputted by the pre-trained machine learning model. The alignment between true oscillatory burst patterns and the predicted probabilities demonstrates that the pre-trained machine learning model is able to capture temporal dependencies and latent structure within neural activity.

FIG. 4C illustrates a validation loss curve produced during the pre-training phase of the neural foundation model. The decreasing validation loss demonstrates a convergence of the pre-training process and indicates that the neural foundation model is learning statistically meaningful structure in the neural token sequences.

FIG. 4D is a bar chart comparing the performance of multiple machine learning models as it pertains to the accuracy of a click task (e.g., a mouse click). The machine learning models assessed include: 1) a baseline model, 2) a simple threshold-based method, 3) a support vector machine (SVM), and 4) the presently-disclosed neural foundation model. The illustrated results demonstrate that the neural foundation model achieves improved balanced accuracy relative to the other approaches, despite being trained with a comparatively small amount of labeled data. The neural foundation model demonstrated increased robustness and accuracy in distinguishing intentional neural commands from unintended neural activity, thereby enabling more reliable real-world neural control. These results indicate that pretraining on neural signals produces transferable representations that improve downstream task performance beyond that achievable with conventional approaches or heuristic approaches. As shown in FIG. 4D, the neural foundation model can be efficiently adapted for real-world use and can outperform alternative machine learning models in practical cognitive decoding applications.

FIG. 5A illustrates one embodiment of a virtual-reality (VR) environment 500 rendered via the VR headset 502. The VR headset 502 can be worn by a subject 114 or user 122 to induce the subject 114 or user 122 to invoke or conjure certain brain states or to elicit certain types of targeted neural activity. The VR headset 502 or a computing device (e.g., the control unit 202 communicatively coupled to the VR headset 502 or a device controlling the VR headset 502) can then record or log contextual data or contextual information concerning the VR environment 500. This contextual data or contextual information can comprise descriptive representations of the VR environment 500 including rendered character(s) 504 or rendered object(s) 506 within the VR environment 500.

In some embodiments, the contextual data or the contextual information obtained from the VR headset 502, or a device communicatively coupled to the VR headset 502, can be labeled (e.g., with text labels, semantic labels, tags, keywords, metadata, numerical labels, key-value pairs, machine-readable data structures, etc.) and such labeled contextual information can be temporally aligned with the sequences of neural signals 112 recorded from the subject 114 or user 122 and provided as inputs to the pre-trained machine learning model 104 as part of the supervised learning phase.

FIG. 5A illustrates a subject 114 or user 122 viewing a rendered character 504 or individual (e.g., a rendered VR person, character, animal, etc.) performing or attempting to perform one or more activities within the VR environment 500. FIG. 5A also illustrates the subject 114 or user 122 viewing or interacting with a rendered object 506 (e.g., a household item, a computer, an appliance, an IoT device, a vehicle, etc.) within the VR environment 500.

In some embodiments, the subject 114 or user 122 can wear the VR headset 502 while performing or attempting to perform the one or more activities themselves or interacting with the rendered object(s) 506 within the VR environment 500. In certain embodiments, the subject 114 or user 122 can use one or more handheld controllers communicatively coupled to the VR headset 502. Also, in these embodiments, the rendered character 504 can be a rendered version (i.e., a VR version) of the subject 114 or user 122. As previously discussed, while the subject or user 122 is wearing the VR headset 502, the subject 114 or user 122 can also have a recording device 118 implanted within the brain of the subject 114 or user 122 or have a number of electrodes 116 positioned or placed on or around the head of the subject 114 or user 122.

The one or more activities can be routine or daily activities that a person would undertake as part of the person's daily life. As such, the VR environment 500 rendered via the VR headset 502 can be an environment familiar to the subject 114 or user 122. In these embodiments, the rendered objects 506 or rendered characters 504 can be objects or characters that the subject 114 or user 122 is familiar with.

For example, the one or more activities can include, but are not limited to, a person (i) reaching for a remote to turn on an entertainment device or television, (ii) operating a phone or other type of mobile device; (iii) turning on a lamp or light, (iv) reaching for a refrigerator to reach a food or beverage from the refrigerator, (v) operating an appliance or IoT device, or (vi) operating or steering a transportation vehicle or a mobility vehicle (e.g., a wheelchair), etc. Although several activities are listed herein, it is contemplated by this disclosure and it should be understood by one of ordinary skill in the art that the list of activities can include other activities not mentioned as part of this disclosure.

In certain embodiments, the one or more activities can include an activity that involves a person moving an appendage (e.g., an arm, a leg, a hand, a foot, a finger, a toe, etc.) to conduct or undertake at least part of the activity. For example, the one or more activities can comprise physical activities such as reaching for an object, holding an object, moving or manipulating an object, turning on or activating an object, etc. Also, for example, the one or more activities can comprise controlling or operating a device 108 or a software application running on the device 108.

In other embodiments, the one or more activities can be new activities that the subject 114 or user 122 is not familiar with or has not undertaken. As such, the VR environment 500 rendered via the VR headset 502 can be an environment that is unfamiliar or new to the subject 114 or user 122. In these embodiments, the rendered objects 506 or rendered characters 504 can be objects or characters that the subject 114 or user 122 is unfamiliar with or is new to the subject 114 or user 122.

In some embodiments, the VR environment 500 can be generated using a world-building ML model to stimulate a physical real-world environment. In some embodiments, the world-building ML model can be run on one or more servers 120 in the cloud or remote servers 120. In other embodiments, the world-building ML model can be run at least partly on the control unit 202. In other embodiments, the world-building ML model can be run on a separate computing device communicatively coupled to the control unit 202. For example, the world-building ML model can be run on the NVIDIA® Omniverse platform. As a more specific example, the world-building ML model can be a generative AI model run on the NVIDIA® Omniverse platform.

The recording device 118 can record sequences of neural signals 112 from the subject 114 or user 122 while the subject 114 or user 122 views the rendered character(s) 504 performing or attempting to perform the one or more activities or views the rendered object(s) 506 within the VR environment 500. The recording device 118 can also record sequences of neural signals 112 from the subject 114 or user 122 while the subject 114 or user 122 interacts or attempts to interact with the rendered character(s) 504, performs or attempts to perform the one or more activities, or interacts or attempts to interact with or operate the rendered object(s) 506 within the VR environment 500.

One technical problem faced by those in the brain-computer interface (BCI) space is how to induce a variety of different brain states of the subject 114 or user 122 or induce different kinds of neural activity such that the recording device 118 is able to record a variety of neural signals reflecting such brain states or neural activity. One technical solution to the aforementioned technical problem is to use the VR headset 502 disclosed herein to render a multitude of scenarios and settings that mimic those in the real-world and allow the subject 114 or user 122 to view activities being performed in these virtual settings or to view rendered objects 506 or rendered characters 504 in these virtual settings. In this manner, the recording device 118 can record the neural signals of the subject 114 or user 122 while the subject 114 or user 122 views activities being performed in these virtual settings or views rendered characters 504 interacting with rendered objects 506 in these virtual settings. This speeds up the post-training process for the pre-trained machine learning model 104 since the subject 114 or user 122 can be shown numerous settings in a limited period of time. This is especially useful if a subject 114 or user 122 is limited in their mobility such as a paraplegic or quadriplegic subject 114 or user 122. Moreover, for a subject 114 or user 122 that is limited in their mobility, the VR headset 502 can allow the subject 114 or user 122 to view a rendered character 504 undertaking a movement or motion or the rendered character 504 interacting with a rendered object 506 or another rendered character 504 in a way that the subject 114 or user 122 might not be able to do in the real-world. While the subject 114 or user 122 is viewing the rendered character 504 undertaking the movement or motion or interacting with the rendered object 506 or rendered character(s) 504, the subject 114 or user 122 can invoke or conjure brain state(s) or elicit neural activity related to the movement or motions, rendered object(s) 506, or rendered character(s) 504 shown via the VR headset 502. This can allow the recording device 118 to record sequences of neural signals 112 of the subject 114 or user 122 while the subject 114 or user 122 is invoking, conjuring, or otherwise generating such brain state(s) or eliciting such neural activity. Without the visual cues provided by the VR environment 500, a subject 114 or user 122 with mobility issues may have difficulties invoking, conjuring, or otherwise generating a reproducible brain state or eliciting reproducible neural activity that relates to a particular motor intention (e.g., reaching for a remote control, reaching for the handle of a refrigerator, turning on a lamp, turning on a dog feeder, turning a steering wheel, etc.), a particular object, or a particular character or person.

Another technical advantage of rendering scenes or settings through a VR environment via the VR headset 502 is that contextual data or contextual information concerning the rendered VR environment 500 can be extracted or otherwise obtained from the VR headset 502 or a computing device controlling or otherwise communicatively coupled to the VR headset 502. This contextual data or contextual information can comprise descriptive representations of the VR environment 500 including rendered character(s) 504 or rendered object(s) 506 within the VR environment 500. As previously discussed, this contextual data or the contextual information obtained or otherwise collected from the VR headset 502 (e.g., via a real-time device log) or a device communicatively coupled to the VR headset 502 can be subsequently labeled and such labeled contextual information 124 can be temporally aligned or synchronized in time with the sequences of neural signals 112 recorded from the subject 114 or user 122.

Contextual data can comprise data or information concerning a context of a scene or environment displayed as part of the VR environment 500 generated by the VR headset 502. As a more specific example, the contextual data can include data or information concerning a setting, a location, a time-of-day, a day-of-the-week, a month, a year, a season, a weather condition, an activity currently being undertaken, one or more individuals rendered in the VR environment 500, and one or more objects or devices rendered in the VR environment 500.

Moreover, as previously discussed, the sequences of neural signals 112 temporally aligned with the labeled contextual information 124 can be provided as inputs to post-train the pre-trained machine learning model 104 as part of a supervised learning phase. As part of the post-training procedure, the pre-trained machine learning model 104 can be provided only with the sequences of neural signals 112 as the inputs and the pre-trained machine learning model 104 can be instructed to output predictions concerning the context shown in the VR environment 500. The pre-trained machine learning model 104 can also receive feedback (e.g., through a reinforcement learning procedure) if the context predicted by the pre-trained machine learning model 104 is different from the contextual data extracted or obtained from the VR environment 500. This can allow the post-trained machine learning model 106 to eventually be able to predict a context of a real-world environment when the post-trained machine learning model 106 is deployed in the real-world and receives as inputs real-time neural signals 132 recorded from a user 122 while the user undertakes activities or encounters objects or individuals in the real-world.

FIG. 5B illustrates one embodiment of an augmented-reality (AR) environment 508 as viewed through an AR wearable 510. The AR wearable 510 can be worn by a subject 114 or user 122 as the subject 114 or user 122 performs or attempts to perform one or more activities or interacts with real object(s) 512 and/or rendered object(s) 514 seen by the subject 114 or user 122.

The one or more activities can be routine or daily activities that a person would undertake as part of the person's daily life. As such, the AR environment 508 can be an environment familiar to the subject 114 or user 122 and the AR wearable 510 can generate rendered object(s) 514 that are familiar to the subject 114 or user 122. In alternative embodiments, the AR wearable 510 can generate rendered object(s) 514 or rendered character(s) that are new or unfamiliar to the subject 114 or user 122.

The AR wearable 510 can render the one or more objects 514 or characters in the AR environment 508 to induce the subject 114 or user 122 to invoke or conjure certain brain states or to elicit certain types of targeted neural activity. In these and other embodiments, the AR wearable 510 can also passively capture an environment surrounding the subject 114 or user 122 including real object(s) 512 or real characters or subjects within the environment. In all such embodiments, the AR wearable 510 or a computing device (e.g., the control unit 202 communicatively coupled to the AR wearable 510 or a device/smartphone controlling the AR wearable 510) can record contextual data or contextual information concerning the AR environment 508 or the real environment surrounding the subject 114 or user 122. This contextual data or contextual information can comprise descriptive representations of the AR environment 508, including rendered character(s) 504 or rendered object(s) 506 and/or the real environment, including real characters or subjects within the real environment.

In some embodiments, the contextual data or the contextual information obtained from the AR wearable 510, or a device communicatively coupled to the AR wearable 510, can be labeled (e.g., with text labels, semantic labels, tags, keywords, metadata, numerical labels, key-value pairs, machine-readable data structures, etc.) and such labeled contextual information 124 can be temporally aligned with the sequences of neural signals 112 recorded from the subject 114 or user 122 and provided as inputs to the pre-trained machine learning model 104 as part of the supervised learning phase.

FIG. 5B illustrates a subject 114 or user 122 viewing the subject 114 or user 122 interacting with a rendered object 514 (e.g., a rendered remote control) within the AR environment 508. While the subject or user 122 is wearing the AR wearable 510, the subject 114 or user 122 can also have a recording device 118 implanted within the brain of the subject 114 or user 122 or have a number of electrodes 116 positioned or placed on or around the head of the subject 114 or user 122.

The recording device 118 can record sequences of neural signals 112 from the subject 114 or user 122 while the subject 114 or user 122 views the rendered objects 514 or characters or interacts/attempts to interact with the rendered objects 514 or characters. Certain objects, settings, or background generated within the AR environment 508 can be generated using the world-building ML model. In some embodiments, the world-building ML model can be run on the same servers 120 used to run the pre-trained machine learning model 104 or the post-trained machine learning model 106. In other embodiments, the world-building ML model can be run on a separate computing device or on a cloud server. For example, the world-building ML model can be run on the NVIDIA® Omniverse platform. As a more specific example, the world-building ML model can be a generative AI model run on the NVIDIA® Omniverse platform.

The recording device 118 can also record sequences of neural signals 112 from the subject 114 or user 122 while the subject 114 or user 122 passively observes the real-world environment surrounding the subject 114 or user 122 (including real-world objects within the environment) via the AR wearable 510 or the subject 114 or user 122 performs routine activities in the real-world while wearing the AR wearable 510. In certain embodiments, the AR wearable 510 can comprise one or more front-facing cameras and/or rear-facing cameras that can capture video recordings of the real-world environment surrounding the subject 114 or user 122. These video recordings can be processed and an object-detection machine learning model or other type of deep learning or computer vision model (running on the control unit 202, a computing device communicatively coupled to the control unit 202, or one or more cloud servers communicatively coupled to the control unit 202 or the computing device) can be used to automatically detect objects or individuals present in the surrounding real-world environment.

One technical problem faced by those in the brain-computer interface (BCI) space is how to induce a variety of different brain states of the subject 114 or user 122 or induce different kinds of neural activity such that the recording device 118 is able to record a variety of neural signals reflecting such brain states or neural activity. One technical solution to the aforementioned technical problem is to use the AR wearable 510 disclosed herein to render a multitude of objects or characters that mimic those in the real-world and allow the subject 114 or user 122 to interact with the rendered objects 514 or rendered characters in the AR environment 508. In this manner, the recording device 118 can record the neural signals of the subject 114 or user 122 while the subject 114 or user 122 interacts with or attempts to interact with the rendered object(s) 514 or rendered characters in the AR environment 508. This speeds up the post-training process for the pre-trained machine learning model 104 since the subject 114 or user 122 can be shown numerous objects or characters in a limited period of time. This is especially useful if a subject 114 or user 122 is limited in their mobility such as a paraplegic or quadriplegic subject 114 or user 122. While the subject 114 or user 122 is viewing or interacting with the rendered object(s) 514 or interacting with the rendered character(s), the subject 114 or user 122 can invoke or conjure brain state(s) or elicit neural activity related to the rendered object(s) 506 or rendered character(s) shown via the AR wearable 510. This can allow the recording device 118 to record sequences of neural signals 112 of the subject 114 or user 122 while the subject 114 or user 122 is invoking, conjuring, or otherwise generating such brain state(s) or eliciting such neural activity. Without the visual cues provided by the AR wearable 510, a subject 114 or user 122 may have difficulties invoking, conjuring, or otherwise generating a reproducible brain state or eliciting reproducible neural activity that relates to such rendered object(s) 506 or rendered characters.

Another technical advantage of generating rendered object(s) 514 or rendered characters via the AR wearable 510 is that data or information concerning the rendered object(s) 514 or the rendered characters can be extracted or otherwise obtained from the AR wearable 510 or a computing device controlling or otherwise communicatively coupled to the AR wearable 510. This contextual data or contextual information can comprise descriptive representations of the rendered object(s) 514 or rendered characters. As previously discussed, this contextual data or the contextual information obtained or otherwise collected from the AR wearable 510 (e.g., via a real-time device log) or a device communicatively coupled to the AR wearable 510 can be subsequently labeled and such labeled contextual information 124 can be temporally aligned or synchronized in time with the sequences of neural signals 112 recorded from the subject 114 or user 122.

Another technical problem faced by those in the brain-computer interface (BCI) space is how to capture contextual data or contextual information concerning the real-world environment surrounding the subject 114 or the user 122. One technical solution to the aforementioned technical problem is to use the AR wearable 510 disclosed herein to capture data and information concerning the real-world environment surrounding the subject 114 or the user 122. As previously discussed, the AR wearable 510 can comprise one or more front-facing cameras and/or rear-facing cameras that can capture video recordings of the real-world environment surrounding the subject 114 or user 122. These video recordings can be processed and an object-detection machine learning model or other type of deep learning or computer vision model (running on the control unit 202, a computing device communicatively coupled to the control unit 202, or one or more cloud servers communicatively coupled to the control unit 202 or the computing device) can be used to automatically detect objects or individuals present in the surrounding real-world environment. Data or information concerning the detected objects and individuals can be stored as contextual data while the subject 114 or user 122 passively observes the real-world environment surrounding the subject 114 or user 122 via the AR wearable 510 or the subject 114 or user 122 performs routine activities in the real-world while wearing the AR wearable 510.

Contextual data can comprise data or information concerning a context of a scene or environment displayed as part of the AR environment 508. As a more specific example, the contextual data can include data or information concerning a setting, a location, a time-of-day, a day-of-the-week, a month, a year, a season, a weather condition, an activity currently being undertaken, and one or more objects or individuals rendered in the AR environment 508.

Moreover, as previously discussed, the sequences of neural signals 112 temporally aligned with the labeled contextual information 124 can be provided as inputs to post-train the pre-trained machine learning model 104 as part of a supervised learning phase. As part of the post-training procedure, the pre-trained machine learning model 104 can be provided only with the sequences of neural signals 112 as the inputs and the pre-trained machine learning model 104 can be instructed to output predictions concerning the context shown in the AR environment 508. The pre-trained machine learning model 104 can also receive feedback (e.g., through a reinforcement learning procedure) if the context predicted by the pre-trained machine learning model 104 is different from the contextual data extracted or obtained from the AR environment 508. This can allow the post-trained machine learning model 106 to eventually be able to predict a context of a real-world environment when the post-trained machine learning model 106 is deployed in the real-world and receives as inputs real-time neural signals 132 recorded from a user 122 while the user undertakes activities or encounters objects or individuals in the real-world.

FIG. 5C illustrates one embodiment of a graphical environment 516 rendered on a screen display 210. In some embodiments, the screen display 210 can be communicatively coupled to the control unit 202 or one or more cloud servers. In other embodiments, the screen display 210 can be controlled by another computing device communicatively coupled to the control unit 202. The screen display 210 can show rendered graphical character(s) 518 performing or attempting to perform one or more activities or interacting with one or more rendered graphical object(s) 520 within the graphical environment 516.

The graphical environment 516 rendered on the screen display 210 can be viewed by the subject 114 or user 122 to induce the subject 114 or user 122 to invoke or conjure certain brain states or to elicit certain types of targeted neural activity. The control unit 202 or another computing device communicatively coupled to the screen display 210 can then record contextual data or contextual information concerning the graphical environment 516. This contextual data or contextual information can comprise descriptive representations of the graphical environment 516 including rendered graphical character(s) 518 or rendered graphical object(s) 520 within the graphical environment 516.

In some embodiments, the contextual data or the contextual information obtained from the control unit 202 or another computing device communicatively coupled to the screen display 210 can be labeled (e.g., with text labels, semantic labels, tags, keywords, metadata, numerical labels, key-value pairs, machine-readable data structures, etc.) and such labeled contextual information 124 can be temporally aligned with the sequences of neural signals 112 recorded from the subject 114 or user 122 and provided as inputs to the pre-trained machine learning model 104 as part of the supervised learning phase.

FIG. 5C illustrates a rendered graphical character 518 (e.g., a graphically rendered person or character) interacting with or attempting to interact with a rendered graphical object 520 (e.g., a graphically rendered remote control). While the subject or user 122 is viewing the graphical environment 516 rendered on the screen display 210, the subject 114 or user 122 can have a recording device 118 implanted within the brain of the subject 114 or user 122 or have a number of electrodes 116 positioned or placed on or around the head of the subject 114 or user 122.

In some embodiments, the subject 114 or user 122 can view the rendered graphical character(s) 518 performing or attempting to perform one or more activities or interacting with the rendered graphical object(s) 520 within the graphical environment 516. The one or more activities can be routine or daily activities that a person would undertake as part of the person's daily life. As such, the graphical environment 516 rendered on the screen display 210 can be an environment familiar to the subject 114 or user 122. In these embodiments, the rendered graphical object(s) 520 or rendered graphical character(s) 518 can be objects or characters that the subject 114 or user 122 is familiar with.

In other embodiments, the one or more activities can be new activities that the subject 114 or user 122 is not familiar with or has not undertaken. As such, the graphical environment 516 rendered on the screen display 210 can be an environment that is unfamiliar or new to the subject 114 or user 122. In these embodiments, the rendered graphical object(s) 520 or rendered graphical character(s) 518 can be objects or characters that the subject 114 or user 122 is unfamiliar with or is new to the subject 114 or user 122.

The recording device 118 can record sequences of neural signals 112 from the subject 114 or user 122 while the subject 114 or user 122 views the rendered graphical character(s) 518 performing or attempting to perform the one or more activities or views the rendered graphical object(s) 520 within the graphical environment 516.

One technical problem faced by those in the brain-computer interface (BCI) space is how to induce a variety of different brain states of the subject 114 or user 122 or induce different kinds of neural activity such that the recording device 118 is able to record a variety of neural signals reflecting such brain states or neural activity. One technical solution to the aforementioned technical problem is to use the screen display 210 disclosed herein to render a multitude of scenarios and settings that mimic those in the real-world and allow the subject 114 or user 122 to view activities being performed in these graphical settings or to view rendered object(s) 520 or rendered character(s) 518 in these graphical settings. In this manner, the recording device 118 can record the neural signals of the subject 114 or user 122 while the subject 114 or user 122 views activities being performed in these graphical settings or views rendered object(s) 520 or rendered character(s) 518 in these graphical settings. This speeds up the post-training process for the pre-trained machine learning model 104 since the subject 114 or user 122 can be shown numerous graphical settings in a limited period of time. This is especially useful if a subject 114 or user 122 is limited in their mobility such as a paraplegic or quadriplegic subject 114 or user 122. While the subject 114 or user 122 is viewing the rendered character(s) 518 undertaking the movement or motion or interacting with the rendered object(s) 520 or rendered character(s) 518, the subject 114 or user 122 can invoke or conjure brain state(s) or elicit neural activity related to the movement or motions, rendered object(s) 520, or rendered character(s) 518 shown on the screen display 210. This can allow the recording device 118 to record sequences of neural signals 112 of the subject 114 or user 122 while the subject 114 or user 122 is invoking, conjuring, or otherwise generating such brain state(s) or eliciting such neural activity. Without the visual cues provided by the graphical environment 516, a subject 114 or user 122 may have difficulties invoking, conjuring, or otherwise generating a reproducible brain state or eliciting reproducible neural activity that relates to a particular motor intention (e.g., reaching for a remote control, reaching for the handle of a refrigerator, turning on a lamp, turning on a dog feeder, turning a steering wheel, etc.), a particular object, or a particular character or person.

Another technical advantage of rendering scenes or settings through the graphical environment 516 is that contextual data or contextual information concerning the rendered graphical environment 516 can be extracted or otherwise obtained from the control unit 202 communicatively coupled to the electronic display 210 or a computing device controlling or otherwise communicatively coupled to the electronic display 210. This contextual data or contextual information can comprise descriptive representations of the graphical environment 516 including rendered character(s) 518 or rendered object(s) 520 within the graphical environment 516. As previously discussed, this contextual data or the contextual information obtained or otherwise collected from the control unit 202 or the computing device controlling the electronic display 210 (e.g., via a real-time device log) can be subsequently labeled and such labeled contextual information 124 can be temporally aligned or synchronized in time with the sequences of neural signals 112 recorded from the subject 114 or user 122.

Contextual data can comprise data or information concerning a context of a scene or environment displayed as part of the graphical environment 516 shown on the electronic display 210. As a more specific example, the contextual data can include data or information concerning a setting, a location, a time-of-day, a day-of-the-week, a month, a year, a season, a weather condition, an activity currently being undertaken, one or more individuals rendered in the graphical environment 516, and one or more objects or devices rendered in the graphical environment 516.

Moreover, as previously discussed, the sequences of neural signals 112 temporally aligned with the labeled contextual information 124 can be provided as inputs to post-train the pre-trained machine learning model 104 as part of a supervised learning phase. As part of the post-training procedure, the pre-trained machine learning model 104 can be provided only with the sequences of neural signals 112 as the inputs and the pre-trained machine learning model 104 can be instructed to output predictions concerning the context shown in the graphical environment 516. The pre-trained machine learning model 104 can also receive feedback (e.g., through a reinforcement learning procedure) if the context predicted by the pre-trained machine learning model 104 is different from the contextual data extracted or obtained from the graphical environment 516. This can allow the post-trained machine learning model 106 to eventually be able to predict a context of a real-world environment when the post-trained machine learning model 106 is deployed in the real-world and receives as inputs real-time neural signals 132 recorded from a user 122 while the user undertakes activities or encounters objects or individuals in the real-world.

FIG. 6 illustrates a user 122 controlling a smart device 600 while wearing one embodiment of a wearable display device 208 to view the smart device 600. In some embodiments, the wearable display device 208 can be an AR wearable 510 such as an AR headset, a pair of smart glasses, or a mixed-reality device. The wearable device 208 can be one example of a portable device 126 (see, e.g., FIG. 1B).

In these embodiments, the smart device 600 can be a smart-home device such as a smart pet feeder or a smart water bowl. The smart device 600 can be considered one of the devices 108 that can be controlled by the systems and method disclosed herein.

The smart device 600 can be a real object 512 seen by the user 122 within the AR environment 508. In addition to the smart device 600, the user 122 can also view real subjects or individuals via the AR environment 508 such as a pet (e.g., a dog) of the user 122.

The wearable display device 208 can continuously capture an external environment surrounding the user 122 including real object(s) 512 or real characters or subjects within the external environment. In these embodiments, the wearable display device 208 or a computing device (e.g., the control unit 202 communicatively coupled to the wearable display device 208 or a device/smartphone controlling the wearable display device 208) can record contextual data or contextual information concerning the external environment surrounding the subject 114 or user 122. This contextual data or contextual information can comprise descriptive representations of the external environment surrounding the user 122 including real characters, subjects, or objects within the external environment. The contextual data or contextual information can be recorded automatically using one or more sensors, system logs, or instrumentation without manual annotation.

FIG. 6 also illustrates that while the user 122 is wearing the wearable display device 208, the user 122 can have a recording device 118 implanted within the brain of the user 122 (or have a number of electrodes 116 positioned or placed on or around the head of the user 122). The recording device 118 can record real-time neural signals 132 from the user 122.

In some embodiments, the real-time neural signals 132 recorded by the recording device 118 can be streamed or otherwise transmitted in real-time to the control unit 202 (e.g., via the telemetry unit 204, see FIG. 2A), a computing device communicatively coupled to the control unit 202, or a cloud server communicatively coupled to the control unit 202. The real-time neural signals 132 can be processed and provided as inputs to the post-trained machine learning model 106 (see, e.g., FIG. 1C to obtain outputs from the post-trained machine learning model 106 in the form of predictions concerning one or more brain states or neural activity of the user 122 reflecting a desire or intention by the user 122 to control the smart device 600.

For example, an intention of the user 122 to control the smart device 600 can be expressed, embodied, or otherwise manifested via one or more brain states or neural activity, or change(s) thereof, invoked, conjured, or otherwise generated by the user 122. The user 122 can generate or invoke or conjure the brain state(s) or neural activity, or change(s) thereof, when the user 122 views the smart device 600 in the real-world via the wearable display device 208.

The post-trained machine learning model 106 can also output the control information 134 needed to control the smart device 600. In certain embodiments, controlling the smart device 600 based on the control information 134 further comprises transmitting device control signals that cause the smart device 600 to perform or cease from performing a physical or operational action. In some embodiments, the post-trained machine learning model 106 can output brain-state embeddings that modulate, gate, delay, or parameterize the control information 134 transmitted to the smart device 600.

In some embodiments, the real-time neural signals 132 can be provided as inputs to the post-trained machine learning model 106 without any real-time contextual information 136 being inputted into the post-trained machine learning model 106 to generate the control information 134. In these embodiments, the post-trained machine learning model 106 is only receiving as inputs the real-time neural signals 132 of the user 122.

In other embodiments, the real-time neural signals 132 can be provided as inputs to the post-trained machine learning model 106 along with real-time contextual information 136 also being inputted into the post-trained machine learning model 106 to generate the control information 134. In these embodiments, the wearable display device 208 (e.g., the AR wearable 510) can comprise at least one front-facing camera 602 and/or one or more rear-facing cameras that can capture video recordings of the real-world or external environment surrounding the user 122. These video recordings can be processed and an object-detection machine learning model or other type of deep learning or computer vision model running on the control unit 202, a computing device communicatively coupled to the control unit 202, or one or more cloud servers communicatively coupled to the control unit 202 or the computing device can be used to automatically detect objects or individuals present in the surrounding real-world environment or external environment. Data or information concerning the detected objects and individuals can be included as part of the real-time contextual information 136 provided as inputs to the post-trained machine learning model 106. The real-time contextual information 136 can be provided as inputs alongside the real-time neural signals 132 to aid the post-trained machine learning model 106 in its inference and generation of the control information 134.

As will be discussed in more detail in relation to FIGS. 7A and 7B, the real-time contextual information 136 can also be used to generate an action list user interface (UI) 700 (see, e.g., FIGS. 7A, 7B, 8A, or 8B) that can be viewed by the user 122 via the wearable display device 208.

Although FIG. 6 illustrates the user 122 controlling the smart device 600 while wearing the wearable display device 208, it should be understood by one of ordinary skill in the art that the user 122 can also wear the wearable display device 208 while going about the daily life of the user 122 or while the user 122 engages in daily activities or routine tasks to allow the system 200 to obtain contextual data or contextual information as part of a post-training phase involving the user 122 (see, e.g., FIG. 1B). For example, the wearable display device 208 can comprise at least one front-facing camera 602 and/or one or more rear-facing cameras that can capture video recordings of the real-world or external environment surrounding the user 122. These video recordings can be processed and an object-detection machine learning model or other type of deep learning or computer vision model running on the control unit 202, a computing device communicatively coupled to the control unit 202, or one or more cloud servers communicatively coupled to the control unit 202 or the computing device can be used to automatically detect objects or individuals present in the surrounding real-world environment or external environment. Data or information concerning the detected objects and individuals can be included as part of the contextual data or the contextual information.

In some embodiments, the contextual data or the contextual information obtained from the wearable display device 208 (or a device controlling the wearable display device 208, the control unit 202, or a computing device communicatively coupled to the control unit 202) can be labeled (e.g., with text labels, semantic labels, tags, keywords, metadata, numerical labels, key-value pairs, machine-readable data structures, etc.) and such labeled contextual information can be temporally aligned with the sequences of neural signals recorded from the user 122 and provided as inputs to the pre-trained machine learning model 104 as part of the supervised learning phase or to post train the pre-trained machine learning model 104.

FIG. 7A illustrates one embodiment of an action list user interface (UI) 700 that can be viewed by the user 122 via the wearable display device 208. The action list UI 700 can comprise a plurality of possible actions 702 that the user 122 can select from the action list UI 700.

As previously discussed, the wearable display device 208 can be an AR wearable 510 or a mixed-reality headset. When the wearable display device 208 is an AR wearable 510, the action list UI 700 can be rendered as part of the AR environment 508 or overlaid as a graphic viewable through the AR wearable 510.

The user 122 can wear the wearable display device 208 to control the device 108 or a software application 110 running on the device 108 based on brain states or neural activity predicted by the post-trained machine learning model 106. An intention of the user 122 to control the device 108 can be expressed, embodied, or otherwise manifested via a brain state or neural activity, or change(s) thereof, invoked, conjured, or otherwise generated by the user 122.

In some embodiments, the user can generate or invoke or conjure the brain state or neural activity, or change(s) thereof when the user 122 views an object 704 in the real-world via the wearable display device 208.

In response to the subject or the user generating, invoking, or conjuring a brain state or neural activity, or change(s) thereof, to interact with or activate the object 704, the recording device 118 can record real-time neural signals 132 associated with or related to the brain state or neural activity, or change(s) thereof.

The real-time neural signals 132 recorded by the recording device 118 can be streamed or otherwise transmitted in real-time to the control unit 202 (e.g., via the telemetry unit 204, see FIG. 2A), a computing device communicatively coupled to the control unit 202, or a cloud server communicatively coupled to the control unit 202. The real-time neural signals 132 can be processed and provided as inputs to the post-trained machine learning model 106 to obtain outputs from the post-trained machine learning model 106 in the form of predictions concerning the control information 134 desired by the user 122.

In some embodiments, the predictions outputted by the post-trained machine learning model 106 concerning the intention(s) or desired control information 134 of the user can be provided as inputs to a generative AI model or another software module to generate at least part of the action list UI 700 to be displayed via the wearable display device 208. In some embodiments, the generative AI model or the other software module can be run on the control unit 202 or another computing device or cloud server 120 communicatively coupled to the control unit 202.

In certain embodiments, the generative AI model or software module can also receive as inputs real-time contextual information 136 obtained from the wearable display device 208, a device or server controlling the wearable display device 208, or another a computing device, tablet, smartphone, or server.

The generative AI model or the software module can generate at least part of the action list UI 700 to be displayed via the wearable display device 208. As shown in FIG. 7A, the action list UI 700 can comprise a plurality of possible actions 702. The plurality of possible actions 702 can reflect the possible control information 134 outputted by the post-trained machine learning model 106 and/or information extracted or otherwise obtained from the real-time contextual information 136.

In some embodiments, the plurality of possible actions 702 can be listed in an order that reflects the confidence level of the predictions outputted by the post-trained machine learning model 106.

As a more specific example, a user 122 wearing the wearable display device 208 (e.g., an AR headset or pair of smart glasses) can see a smart lamp (e.g., a lamp that can be controlled via Wi-Fi or Bluetooth®) sitting on a table nearby. The user 122 can invoke or conjure one or more brain state(s) or neural activity related to turning on the smart lamp. The recording device 118 can stream or transmit the real-time neural signals 132 recorded from the user 122 to the control unit 202 or a computing device communicatively coupled to the control unit 202. The real-time neural signals 132 (or filtered/processed instances of the real-time neural signals 132) can be provided as inputs to the post-trained machine learning model 106. The post-trained machine learning model 106 can output a number of possible control information 134 based on the one or more brain state(s) or neural activity of the user 122. The predictions outputted by the post-trained machine learning model 106 can then be provided as inputs to the generative AI model to generate the action list UI 700 shown in FIG. 7A.

FIG. 7B illustrates the user 122 selecting one of the plurality of possible actions 702 from the action list UI 700. For example, as shown in FIG. 7B, the user can select “Turn on the lamp” from the list of possible actions 702 from the action list UI 700.

In some embodiments, the selection from the user 122 can be received by processing further real-time neural signals 132 recorded by the recording device 118. For example, the user, after being presented with the action list UI 700, can evoke or conjure one or more new brain state(s) or new neural activity concerning the possible action 702 desired by the user. This new brain state or new neural activity can then be picked up by the recording device 118 via the real-time neural signals 132 recorded from the user 122.

In other embodiments, the selection from the user 122 can be received via an eye tracking device 706. In these embodiments, the eye tracking device 706 can be integrated with the wearable display device 208. In other embodiments, the eye tracking device can be a standalone eye tracking device. In additional embodiments, the selection from the user 122 can be received via voice recognition software or via one or more handheld controllers operated by the user 122.

Once the control unit 202, or a computing device or cloud server communicatively coupled to the control unit 202, has received the selection from the user 122 concerning one of the possible actions 702, the control unit 202, or the computing device or cloud server communicatively coupled to the control unit 202, can control the device 108 or a software application running on the device 108 by transmitting the control information 134. As previously discussed, in some embodiments, the control unit 202, or a computing device or cloud server communicatively coupled to the control unit 202, can control the device 108 or a software application running on the device 108 by transmitting the control information 134 directly to the device 108 or the software application running on the device 108. In additional embodiments, the control unit 202, or a computing device or cloud server communicatively coupled to the control unit 202, can control the device 108 or a software application running on the device 108 by transmitting the commands to an AI software agent or a robotic component (e.g., a robotic arm or a general-purpose robot) for carrying out or executing the control information 134.

FIG. 8A illustrates another embodiment of an action list UI 700 concerning whether to clean up pet food that has spilled onto the ground from an automated pet feeder. FIG. 8A illustrates that real-time contextual information 136 in the form of text-based labels, tags, or other types of semantic information can be provided as inputs to the post-trained machine learning model 106 along with real-time neural signals 132 recorded from the user 122 in order to generate a plurality of possible actions 702 of an action list UI 700. Each of the plurality of possible actions 702 can be associated with a particular control information 134 for controlling a device 108 such as the smart vacuum cleaner shown in FIG. 8A.

As previously discussed, the wearable display device 208 can comprise at least one front-facing camera 602 (and/or one or more rear-facing cameras) that can capture video recordings of the real-world or external environment surrounding the user 122. These video recordings can be filtered and processed and an object-detection machine learning model or other type of deep learning or computer vision model running on the control unit 202, a computing device communicatively coupled to the control unit 202, or one or more cloud servers communicatively coupled to the control unit 202 or the computing device can be used to automatically detect objects or individuals present in the surrounding real-world environment or external environment. Data or information concerning the detected objects and individuals can be included as part of the real-time contextual information 136. The real-time contextual information 136 can be provided as inputs alongside the real-time neural signals 132 to aid the post-trained machine learning model 106 in its inference and generation of the plurality of possible actions 702 and the control information 134 associated with the plurality of possible actions 702.

When the wearable display device 208 is an AR wearable 510, the action list UI 700 can be rendered as part of the AR environment 508 or overlaid as a graphic viewable through the AR wearable 510.

As a more specific example, a user 122 wearing the wearable display device 208 (e.g., an AR headset or pair of smart glasses) can see a pet feeder with pet food spilled onto the ground near a smart vacuum (e.g., a vacuum that can be controlled via Wi-Fi or Bluetooth®). The user 122 can invoke or conjure one or more brain state(s) or neural activity related to cleaning up the spilled pet food or turning on the vacuum. The recording device 118 can stream or transmit the real-time neural signals 132 recorded from the user 122 to the control unit 202 or a computing device communicatively coupled to the control unit 202.

At around the same time, the front-facing camera 602 (e.g., see FIG. 6) of the wearable display device 208 can capture video recordings where the video frames of such video recordings show the smart vacuum, the pet feeder, and the spilled dog food. These video frames contained within the video recordings can be filtered and processed and an object-detection machine learning model or other type of deep learning or computer vision model can detect the smart vacuum, the pet feeder, and the spilled pet food as objects within the video frames. Data or information concerning the detected objects can be included as part of the real-time contextual information 136. The real-time contextual information 136 can be provided as inputs alongside the real-time neural signals 132 or filtered/processed instances of the real-time neural signals 132) to aid the post-trained machine learning model 106 in its inference and generation of the plurality of possible actions 702 and the control information 134 associated with the plurality of possible actions 702.

The post-trained machine learning model 106 can output a number of possible control information 134 based on the one or more brain state(s) or neural activity of the user 122 and the real-time contextual information 136. In some embodiments, the predictions outputted by the post-trained machine learning model 106 and the real-time contextual information 136 can then be provided as inputs to a generative AI model to generate the action list UI 700 shown in FIG. 8A. In other embodiments, the predictions outputted by the post-trained machine learning model 106 and the real-time contextual information 136 can be provided as inputs to a software module to render the action list UI 700.

FIG. 8A also illustrates the user 122 selecting one of the plurality of possible actions 702 from the action list UI 700. For example, as shown in FIG. 8A, the user can select “30 minute clean” from the list of possible actions 702 from the action list UI 700 to transmit one or more commands or control signals to the smart vacuum to clean the area near the pet feeder for 30 minutes.

In some embodiments, the selection from the user 122 can be received by processing further real-time neural signals 132 recorded by the recording device 118. For example, the user, after being presented with the action list UI 700, can evoke or conjure one or more new brain state(s) or new neural activity concerning the possible action 702 desired by the user. This new brain state or new neural activity can then be picked up by the recording device 118 via the real-time neural signals 132 recorded from the user 122.

In other embodiments, the selection from the user 122 can be received via the eye tracking device 706 integrated with the wearable display device 208 or be received via a standalone eye tracking device. In additional embodiments, the selection from the user 122 can be received via voice recognition software or via one or more handheld controllers operated by the user 122.

FIG. 8B illustrates another embodiment of an action list UI 700 concerning whether to turn on a fan. FIG. 8B illustrates that real-time contextual information 136 in the form of text-based labels, tags, or other types of semantic information can be provided as inputs to the post-trained machine learning model 106 along with real-time neural signals 132 recorded from the user 122 in order to generate a plurality of possible actions 702 of an action list UI 700. Each of the plurality of possible actions 702 can be associated with a particular control information 134 for controlling a device 108 such as the smart fan (e.g., a fan that can be controlled via Wi-Fi or Bluetooth® or via a Wi-Fi or Bluetooth® power plug).

As previously discussed, the wearable display device 208 can comprise at least one front-facing camera 602 (and/or one or more rear-facing cameras) that can capture video recordings of the real-world or external environment surrounding the user 122. These video recordings can be processed and an object-detection machine learning model or other type of deep learning or computer vision model running on the control unit 202, a computing device communicatively coupled to the control unit 202, or one or more cloud servers communicatively coupled to the control unit 202 or the computing device can be used to automatically detect objects or individuals present in the surrounding real-world environment or external environment. Data or information concerning the detected objects and individuals can be included as part of the real-time contextual information 136. The real-time contextual information 136 can be provided as inputs alongside the real-time neural signals 132 to aid the post-trained machine learning model 106 in its inference and generation of the plurality of possible actions 702 and the control information 134 associated with the plurality of possible actions 702. Moreover, the post-trained machine learning model 106 can also receive as inputs additional contextual data or information concerning a temperature within the room, the current season, a weather or climate report, etc.

When the wearable display device 208 is an AR wearable 510, the action list UI 700 can be rendered as part of the AR environment 508 or overlaid as a graphic viewable through the AR wearable 510.

As a more specific example, a user 122 wearing the wearable display device 208 (e.g., an AR headset or pair of smart glasses) can see the smart fan. The user 122 can invoke or conjure one or more brain state(s) or neural activity related to turning on or adjusting a speed of the smart fan. The recording device 118 can stream or transmit the real-time neural signals 132 recorded from the user 122 to the control unit 202 or a computing device communicatively coupled to the control unit 202. The real-time neural signals 132 (or filtered/processed instances of the real-time neural signals 132) can be provided as inputs to the post-trained machine learning model 106 along with the real-time contextual information 136. The post-trained machine learning model 106 can output a number of possible control information 134 based on the one or more brain state(s) or neural activity of the user 122. In some embodiments, the predictions outputted by the post-trained machine learning model 106 can then be provided as inputs along with the real-time contextual information 136 to a generative AI model to generate the action list UI 700 shown in FIG. 8A. In other embodiments, the predictions outputted by the post-trained machine learning model 106 can then be provided as inputs along with the real-time contextual information 136 to a software module to render the action list UI 700.

FIG. 8B also illustrates the user 122 selecting one of the plurality of possible actions 702 from the action list UI 700. For example, as shown in FIG. 8A, the user can select “Fan high” from the list of possible actions 702 from the action list UI 700 to transmit one or more commands or control signals to the smart fan to turn the fan onto its highest setting.

In some embodiments, the selection from the user 122 can be received by processing further real-time neural signals 132 recorded by the recording device 118. For example, the user, after being presented with the action list UI 700, can evoke or conjure one or more new brain state(s) or new neural activity concerning the possible action 702 desired by the user. This new brain state or new neural activity can then be picked up by the recording device 118 via the real-time neural signals 132 recorded from the user 122.

In other embodiments, the selection from the user 122 can be received via an eye tracking device 706. In these embodiments, the eye tracking device 706 can be integrated with the wearable display device 208. In other embodiments, the eye tracking device can be or refer to a standalone eye tracking device. In additional embodiments, the selection from the user 122 can be received via voice recognition software or via one or more handheld controllers operated by the user 122.

In some embodiments, the real-time contextual information 136 obtained from the wearable display device 208 (e.g., the AR wearable) can be provided as inputs to the post-trained machine learning model 106 to assist in generating, filtering, ranking, or otherwise structuring an action list UI 700 presented to the user 122.

In these embodiments, the user 122 can wear a wearable display device 208 that captures real-time contextual information 136 describing an environment surrounding the user 122, such as detected objects, object locations, device states, user gaze direction, spatial relationships, or interaction history. The wearable display device 208 (e.g., AR wearable) can comprise one or more cameras, depth sensors, eye tracking device(s) 706, or other sensors configured to generate contextual representations of the scene surrounding the user 122.

While object-detection or scene-understanding modules can initially identify candidate objects or devices within the environment, the post-trained machine learning model 106 can receive real-time neural signals 132 from the user 122 and real-time contextual information 136 from the wearable display device 208. Based on the inferred brain state of the user 122 and the real-time contextual information 136 describing the environment, the post-trained machine learning model 106 can generate control information 134 usable to determine which actions are relevant, appropriate, or likely intended by the user 122 at that moment.

For example, a user 122 wearing AR goggles can be visually observing a smart lamp, a smart fan, and a television within the same room. Object-detection systems can identify all three devices as candidates for interaction. However, the user's real-time neural signals 132 can reflect a brain state associated with thermal discomfort, restlessness, or cooling-related intent. When the post-trained machine learning model 106 receives both the real-time neural signals 132 and the real-time contextual information 136 indicating the presence and location of the smart fan, the post-trained machine learning model 106 can generate control information 134 that prioritizes fan-related actions (e.g., “Turn fan on” or “Increase fan speed”) while deprioritizing or excluding unrelated actions associated with the other detected devices.

In this manner, the action list UI 700 displayed via the AR goggles is shaped not solely by object detection, but by brain-state-conditioned interpretation of context, enabling the action list UI 700 to reflect the inferred intent, cognitive state, or situational goals of the user 122 rather than presenting all possible actions 702 uniformly.

In some embodiments, the post-trained machine learning model 106 can generate control information 134 that filters candidate actions derived from contextual object detection, ranks candidate actions based on inferred intent or confidence, suppresses actions inconsistent with the inferred brain state, introduces actions not explicitly tied to a single object (e.g., “Cool the room,” “Pause activity,” or “Reduce stimulation”), or conditions whether an action list UI 700 is displayed at all.

In further embodiments, the post-trained machine learning model 106 can operate in conjunction with downstream software modules or user interface engines, such that the model does not directly render the action list UI 700 but instead outputs brain-state-conditioned control information 134 that governs how the action list UI 700 is generated, ordered, or presented.

This embodiment demonstrates that real-time contextual information 136 obtained from a wearable display device 208 (e.g., the AR wearable) can be used not only to identify objects in an environment, but also to enable brain-state-aware action list generation, thereby improving relevance, reducing cognitive load, and supporting more intuitive brain-driven interaction with complex environments.

FIG. 9 illustrates non-limiting examples of brain states corresponding to reproducible neural activity that can be detected in different regions of the brain. A brain state can be defined operationally by its neural characteristics, such as a spatial distribution, a temporal dynamic, or a frequency content or distribution of neural activity. For example, a brain state can correspond to neural activity or neural patterns associated with perception, cognition, emotion, or motor planning. A brain state can also be represented by intermediate or latent neural configurations. Brain states can be viewed as cognitive primitives or foundational units of neural representation from which higher-order cognitive, behavioral, or control-related functions can be inferred.

In some embodiments, neural signals can be recorded using the recording device 118 and then discretized into sequences of brain states. Each brain state can function as a cognitive primitive representing a functionally distinct pattern of distributed brain activity. These brain states can then be inputted into the post-trained machine learning model 106 to infer brain state transitions or embeddings and generate control information 134 for device control.

For example, neural signals can be recorded from electrodes 116 implanted in one or more subjects 114 across multiple cortical and/or subcortical regions the one or more subjects 114 while the one or more subjects 114 engage in natural behavior and everyday activities.

The recorded neural signals can include time-varying neural activity spanning motor, sensory, associative, and limbic regions of the brain. Prior to associating the neural signals with task-specific labels, the neural signals can be discretized into sequences of brain states. Each brain state can represent a structured pattern of neural activity distributed across multiple recording locations within the brain and time windows. The brain states can encode spatial, temporal, and frequency-related characteristics of neural activity, rather than isolated channel events.

Brain states can correspond to distributed neural patterns that, in some embodiments, are associated with observable behaviors or internal conditions of the subject(s) 114. These can include detecting an error while typing or realizing a mispronounced word, adjusting grip strength when holding a fragile object, planning or initiating a motor action such as moving a computer mouse or pressing a brake pedal, recognizing a familiar face or place, processing linguistic structure such as detecting sarcasm or identifying a grammatical inconsistency, refocusing attention after mental drift, maintaining motivation or emotional regulation during challenging situations, detecting surprise, embarrassment, or emotional salience, or processing auditory patterns such as recognizing a song or detecting an off note. It should be understood by one of ordinary skill in the art that these examples are illustrative of how brain states can manifest in practice, but do not limit the definition of brain states to any particular cognitive, emotional, or behavioral category.

The machine learning model 102 (e.g., a neural foundation model) can be pre-trained using unsupervised or self-supervised learning (see, e.g., FIG. 1A) on sequences of brain states. In one embodiment, pre-training is performed using an autoregressive objective in which the machine learning model 102 predicts future brain states based on preceding brain states. In other embodiments, masked prediction, contrastive learning, or hybrid self-supervised objectives can also be used as part of the pre-training phase.

During this pre-training phase, the machine learning model 102 learns latent representations that capture structure and relationships across sequences of brain states in a task-agnostic manner, without requiring labels corresponding to specific actions, emotions, or commands. Following the pre-training phase, the pre-trained machine learning model 104 can be post-trained using neural signals temporally aligned with contextual information (e.g., labeled contextual information 124). For example, the contextual information can be obtained by passively observing one or more environments or activities of the subject(s) 114 or user(s) 122 (e.g., by recording, capturing, or collecting audio, video(s), text data or semantic information, device usage patterns, etc.) or by intentionally modifying the environment(s) of the subject(s) 114 or user(s) 122 induce particular brain states. In this example, contextual information can be used to shape and disambiguate brain state representations corresponding to the distributed neural patterns associated with the observable behaviors or conditions shown in FIG. 9.

As a more specific example, contextual cues or contextual information indicating a typing task can be aligned with brain states corresponding to error detection or corrective planning. Also, as another example, contextual cues or contextual information indicating a virtual task or physical task requiring fine motor control can be aligned with brain states corresponding to grip adjustment or coordinated movement. As an additional example, contextual cues or contextual information indicating a conversation can be aligned with brain states corresponding to linguistic or social processing.

During the post-training phase, this temporally aligned contextual information is used as supervisory signals to adapt the pretrained representations of the pre-trained machine learning model 104, improving separability and robustness of the model while preserving the task-agnostic structure learned during the pre-training phase.

After the post-training phase, real-time neural signals recorded from the user are provided as inputs to the post-trained machine learning model 106. In some embodiments, model inference is performed using neural signals alone, without requiring contextual inputs at runtime.

The post-trained machine learning model 106 can process real-time neural signals representing sequences of brain states and infer one or more current or evolving brain states of the user. In this sense, the inferred brain states can operate as cognitive primitives that distinguish neural activity associated with intentional control from neural activity associated with background cognition, perception, or emotion.

Based on the inferred brain states, the post-trained machine learning model 106 can output control information 134. The control information 134 can be discrete or continuous. In some embodiments, the control information 134 can be generated directly by the post-trained machine learning model 106 or by a downstream mapping module. The control information 134 generated by the post-trained machine learning model 106 can be used to control one or more electronic devices 108. Control information 134 can be generated that control a computing device, select interface elements, actuate a mechanical component, or initiate or inhibit operation of an electronic subsystem. The control information 134 can include delays, confirmations, suppressions, prioritizations, or parameter adjustments, rather than simple on/off commands.

Since the post-trained machine learning model 106 operates on learned representations of brain states acting as cognitive primitives, rather than task-specific decoders, the same model can be reused across multiple devices, tasks, and users with minimal additional training. This reinforces that the systems and methods disclosed herein operate at a brain-state-to-control abstraction layer, rather than a task-specific decoder, preserving scalability and generality.

The following are examples that illustrate non-limiting associations between inferred brain states and corresponding device control behavior:

The post-trained machine learning model 106 can infer a brain state of the user 122 associated with cognitive control or decision-making such as hesitations or concern related to sending a risky text message. In this example scenario, the control information 134 outputted can cause a text messaging application to pause transmission or present a confirmation prompt indicating elevated risk, optionally requiring an explicit confirmation or delay before sending the risky text message.

The post-trained machine learning model 106 can infer a brain state of the user 122 associated with reflecting on past decisions or imagining different outcomes. In this example scenario, the control information 134 outputted can delay irreversible actions (e.g., deletion, submission, purchase) and optionally log the event for later review rather than executing immediately.

The post-trained machine learning model 106 can infer a brain state of the user 122 associated with planning out life choices or setting a goal. In this example scenario, the control information 134 outputted can trigger creation of a reminder, note, or planning interface instead of executing an immediate action.

The post-trained machine learning model 106 can infer a brain state of the user 122 associated with deciding not to make a same or similar mistake that was made in the past. In this example scenario, the control information 134 outputted can suppress execution of a previously repeated action and can also surface alternative options.

The post-trained machine learning model 106 can infer a brain state of the user 122 associated with attention, focus or error detection such as detecting an error while typing or realizing a mispronounced word. In this example scenario, the control information 134 outputted can automatically highlight text, pause inputs, or offer a correction before continuing.

The post-trained machine learning model 106 can infer a brain state of the user 122 associated with a desire to refocus after drifting off in a daydream. In this example scenario, the control information 134 outputted can restore task focus, resume paused content, or remove a background distraction.

The post-trained machine learning model 106 can infer a brain state of the user 122 associated with knowing a sentence is grammatically wrong. In this example scenario, the control information 134 outputted can flag text for review or correction prior to submission.

The post-trained machine learning model 106 can infer a brain state of the user 122 associated with understanding a metaphor or detecting sarcasm. In this example scenario, the control information 134 outputted can adjust downstream interpretation (e.g., sentiment analysis, response tone) rather than executing a literal action.

The post-trained machine learning model 106 can infer a brain state of the user 122 associated with motor planning and physical interactions such as a desire to move a computer mouse. In this example scenario, the control information 134 outputted can initiate a click command, increase cursor sensitivity, or enable continuous cursor motion control.

The post-trained machine learning model 106 can infer a brain state of the user 122 associated with a desire to type on a keyboard. In this example scenario, the control information 134 outputted can initiate a keyboard press, accelerate text input, auto-complete, or initiate predictive typing.

The post-trained machine learning model 106 can infer a brain state of the user 122 associated with multi-tasking motor activities. In this example scenario, the control information 134 outputted can prioritize or multiplex control signals across multiple device functions.

The post-trained machine learning model 106 can infer a brain state of the user 122 associated with desire to adjust grip strength when holding something fragile. In this example scenario, the control information 134 outputted can reduce an actuation force or constrain a movement of a robotic or prosthetic device.

The post-trained machine learning model 106 can infer a brain state of the user 122 associated with a desire to press down on the brake pedal. In this example scenario, the control information 134 outputted can trigger immediate braking or inhibit conflicting commands.

The post-trained machine learning model 106 can infer a brain state of the user 122 associated with feeling embarrassed. In this example scenario, the control information 134 outputted can suppress broadcasting, sharing, or recording actions.

The post-trained machine learning model 106 can infer a brain state of the user 122 associated with feeling surprised or shocked. In this example scenario, the control information 134 outputted can temporarily pause automated actions to prevent unintended execution.

The post-trained machine learning model 106 can infer a brain state of the user 122 associated with feeling motivated to push through a challenge. In this example scenario, the control information 134 outputted can reduce friction (e.g., by reducing fewer confirmations and enabling faster execution) for task-relevant actions.

The post-trained machine learning model 106 can infer a brain state of the user 122 associated with staying calm in an argument. In this example scenario, the control information 134 outputted can limit reactive responses, delay message sending, or enforce cooling-off intervals.

The post-trained machine learning model 106 can infer a brain state of the user 122 associated with memory and recognition such as recognizing someone familiar. In this example scenario, the control information 134 outputted can automatically bring up contextual information (e.g., name suggestions) without automatically initiating interaction.

The post-trained machine learning model 106 can infer a brain state of the user 122 associated with recognizing a familiar place. In this example scenario, the control information 134 outputted can retrieve location-relevant information or settings.

The post-trained machine learning model 106 can infer a brain state of the user 122 associated with remembering a face (but not a name). In this example scenario, the control information 134 outputted can prompt recall aids without forcing identification.

The post-trained machine learning model 106 can infer a brain state of the user 122 associated with recognizing the cry of one's child in a noisy environment. In this example scenario, the control information 134 outputted can automatically turn off any audio or video being played and reduce any incoming sounds or distractions.

The post-trained machine learning model 106 can infer a brain state of the user 122 associated with sensory recognition or recall such as recognizing a song. In this example scenario, the control information 134 outputted can retrieve or display metadata concerning the song rather than changing a playback state.

The post-trained machine learning model 106 can infer a brain state of the user 122 associated with detecting an off note in a musical performance. In this example scenario, the control information 134 outputted can flag for further audio analysis or turn on recording for further review.

The post-trained machine learning model 106 can infer a brain state of the user 122 associated with a smell remembered from one's childhood. In this example scenario, the control information 134 outputted can log or bookmark contextual data associated with the smell.

The post-trained machine learning model 106 can infer a brain state of the user 122 associated with behavioral inhibition or regulation such as stopping oneself from laughing in a serious moment. In this example scenario, the control information 134 outputted can suppress expressive outputs (e.g., emojis, voice modulation).

The post-trained machine learning model 106 can infer a brain state of the user 122 associated with stopping oneself from reaching for the phone when a need to focus arises. In this example scenario, the control information 134 outputted can lock the phone or restrict phone usage temporarily.

The post-trained machine learning model 106 can infer a brain state of the user 122 associated with a desire to stop eating when feeling full. In this example scenario, the control information 134 outputted can inhibit online ordering, online purchasing, or reminder actions related to food.

One technical problem faced by those in the brain-computer interface (BCI) space is that conventional machine learning models used by BCI systems suffer from fundamental technical limitations that prevent the scalability of such BCI systems across tasks, environments, and users. Such conventional machine learning models typically rely on task-specific or supervised decoding pipelines that require predefined labels, hand-designed features, and per-user calibration, resulting in models that generalize poorly beyond the narrow conditions under which they were trained. Contextual information, when used at all, is treated as static metadata or is required continuously at inference, creating brittle BCI systems that degrade when sensors, environments, or user behaviors change. Such conventional machine learning models also depend on labor-intensive data labeling and yield low signal separability when relying solely on passive neural observation, making it difficult to reliably capture diverse cognitive or brain states at scale. As a result, current BCI systems do not support efficient population-level training, rapid onboarding of new users, or reuse of learned representations across devices or applications. One technical solution to the aforementioned technical problem is the method disclosed herein that overcomes these scalability barriers by decoupling neural representation learning from task definitions and device-specific controls. Rather than training a machine learning model to decode predefined tasks or commands, the systems and methods disclosed herein rely on a neural foundation model that first learns task-agnostic latent representations of neural activity from sequences of neural signals during an unsupervised or self-supervised learning phase. Contextual information, either derived from passive observation of a subject's environment or from active modification of that environment, can then be used during a post-training phase to shape and disambiguate these latent representations, improving robustness and separability. Once trained in this manner, the neural foundation model can infer brain states or intent from the user's neural signals and generate control information that can be sent to control one or more devices or software applications running on such devices. This functional separation enables the neural foundation model to be reused across multiple tasks, environments, and users, supporting scalable deployment without repeated task-specific retraining.

Another technical solution to the aforementioned technical problem is the system disclosed herein having a multi-stage neural processing architecture centered on a neural foundation model trained on neural signal sequences recorded from implanted and/or non-invasive electrodes. The architecture comprises a pre-training phase configured to perform unsupervised or self-supervised learning on unlabeled neural data, followed by a post-training stage in which neural signals are temporally aligned with contextual information obtained from external sensing or devices/systems that modify a simulated environment. Context generation and acquisition components can include passive sensing pipelines or immersive physical, virtual, or augmented environments designed to elicit specific brain states. The post-trained neural foundation model converts inferred brain states into control information, which interface with device control logic. This modular structure separates data acquisition, representation learning, contextual alignment, inference, and device control, enabling scalable training, flexible inference configurations, and reuse of learned representations across different devices and application domains.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

The term “engine” or “module” as used herein can refer to software, firmware, hardware, or a combination thereof. In the case of a software implementation, for instance, these may represent program code that performs specified tasks when executed on a processor (e.g., CPU, GPU, or processor cores therein). The program code can be stored in one or more computer-readable memory or storage devices. Any references to a function, task, or operation performed by an “engine” or “module” can also refer to one or more processors of a device or server programmed to execute such program code to perform the function, task, or operation.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A method of controlling one or more devices, comprising:
pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model;
post-training the pre-trained machine learning model using supervised learning on the sequences of neural signals or additional sequences of neural signals aligned with labeled contextual information to yield a post-trained machine learning model, wherein the labeled contextual information is obtained by recording contextual data using one or more modalities in real or simulated environments experienced by the one or more subjects or one or more users, wherein the labeled contextual information comprises descriptive representations derived from the one or more modalities obtained during at least one of:
passively observing an environment or activities of the one or more subjects or the one or more users, and
modifying the environment of the one or more subjects or the one or more users to induce targeted neural activity, wherein, during the post-training, the pre-trained machine learning model transforms the task-agnostic latent representations learned during the pre-training into brain-state embeddings configured to align neural and contextual representations;
inputting real-time neural signals recorded from one of the users into the post-trained machine learning model to generate control information based on the brain-state embeddings; and
controlling one or more devices based on the control information.

2. The method of claim 1, wherein the machine learning model is a neural foundation model.

3. The method of claim 1, wherein the task-agnostic latent representations are internal representations learned by the machine learning model during the pre-training that encode structure, patterns, and relationships present in the sequences of neural signals without relying on the task-specific labels or predefined outputs, wherein the task-agnostic latent representations are distinct from raw neural signals, device control commands, or the task-specific labels.

4. The method of claim 1, wherein pre-training the machine learning model further comprises intentionally excluding task labels associated with the sequences of neural signals.

5. The method of claim 1, wherein pre-training the machine learning model is performed without requiring the machine learning model to output a task identifier, a command label, or a device control action.

6. The method of claim 1, wherein pre-training the machine learning model further comprises providing the sequences of neural signals recorded from the electrodes as inputs to the machine learning model and obtaining, as outputs, autoregressive predictions, masked neural signal predictions, or contrastive learning predictions of upcoming or ensuing neural signals.

7. The method of claim 6, wherein the sequences of neural signals provided as inputs to the machine learning model are provided as discretized neural signals treated as tokenized inputs.

8. The method of claim 1, wherein passively observing the environment or the activities of the one or more subjects or the one or more users further comprises observing the environment or the activities of the one or more subjects or the one or more users without intentionally modifying the environment of the one or more subjects or the one or more users or interfering with the activities of the one or more subjects or the one or more users, and wherein recording the contextual data using the one or more modalities further comprises recording data or information concerning a physical environment, a virtual environment, or an augmented environment experienced by the one or more subjects or the one or more users.

9. The method of claim 1, wherein modifying the environment of the one or more subjects or the one or more users further comprises intentionally modifying a virtual environment or an augmented environment of the one or more subjects or the one or more users to induce the targeted neural activity, and wherein recording the contextual data using the one or more modalities further comprises recording modifications to the virtual environment or the augmented environment of the one or more subjects or the one or more users.

10. The method of claim 9, wherein intentionally modifying the environment of the one or more subjects or the one or more users further comprises:
intentionally modifying the virtual environment of the one or more subjects or the one or more users via a virtual reality device worn by one of the subjects or one of the users, or
intentionally modifying the augmented environment of the one or more subjects or the one or more users via an augmented reality device worn by one of the subjects or one of the users.

11. The method of claim 1, wherein the contextual data is recorded automatically using one or more sensors, system logs, or instrumentation without manual annotation.

12. The method of claim 1, wherein inputting the real-time neural signals into the post-trained machine learning model is undertaken without any real-time contextual information being inputted into the post-trained machine learning model to generate the control information.

13. The method of claim 1, wherein inputting the real-time neural signals into the post-trained machine learning model is undertaken with real-time contextual information also being inputted into the post-trained machine learning model to generate the control information.

14. The method of claim 1, wherein the sequences of neural signals are recorded from the electrodes implanted within or positioned on the one or more subjects.

15. The method of claim 1, wherein the post-trained machine learning model further comprises one or more classifiers, one or more decoder heads, or a combination thereof.

16. The method of claim 1, wherein the post-trained machine learning model is able to be used to accomplish different tasks without having to re-train the machine learning model.

17. The method of claim 1, wherein the post-trained machine learning model is able to be to be used by different users without having to re-train the machine learning model.

18. The method of claim 1, wherein controlling the one or more devices based on the control information further comprises transmitting device control signals that cause the one or more devices to perform or cease from performing a physical or operational action.

19. The method of claim 1, wherein the brain-state embeddings modulate, gate, delay, or parameterize the control information.

20. The method of claim 1, wherein the one or more devices comprises at least one of a computing device, a robotic device, an assistive device, a smart-home device, an Internet-of-Things (IoT) device, and a mobility vehicle.

21. A method of controlling one or more devices, comprising:
providing a pre-trained machine learning model;
post-training the pre-trained machine learning model using supervised learning on sequences of neural signals aligned with labeled contextual information to yield a post-trained machine learning model, wherein the labeled contextual information is obtained by recording contextual data from one or more modalities in real or simulated environments experienced by one or more subjects or one or more users, wherein the labeled contextual information comprises descriptive representations derived from the one or more modalities obtained during at least one of:
passively observing an environment or activities of the one or more subjects or the one or more users, and
modifying the environment of the one or more subjects or the one or more users to induce targeted neural activity, wherein, during the post-training, the pre-trained machine learning model transforms task-agnostic latent representations learned during pre-training into brain-state embeddings configured to align neural and contextual representations;
inputting real-time neural signals recorded from one of the users into the post-trained machine learning model to generate control information based on the brain-state embeddings; and
controlling one or more devices based on the control information.

22.-40. (canceled)

41. A method of controlling one or more devices, comprising:
inputting real-time neural signals recorded from a user into a post-trained machine learning model to generate control information based on brain-state embeddings,
wherein the post-trained machine learning model is obtained by:
pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model, and
post-training the pre-trained machine learning model using supervised learning on the sequences of neural signals or additional sequences of neural signals aligned with labeled contextual information to yield the post-trained machine learning model, wherein the labeled contextual information is obtained by recording contextual data from one or more modalities in real or simulated environments experienced by the one or more subjects or one or more users, wherein the labeled contextual information comprises descriptive representations derived from the one or more modalities obtained during at least one of:
passively observing an environment or activities of the one or more subjects or the one or more users, and
modifying the environment of the one or more subjects or the one or more users to induce targeted neural activity, wherein, during the post-training, the pre-trained machine learning model transforms the task-agnostic latent representations learned during the pre-training into the brain-state embeddings configured to align neural and contextual representations; and
controlling one or more devices based on the control information.

42.-60. (canceled)

61. A system for controlling one or more devices, comprising:
a recording device configured to record real-time neural signals of a user; and
a control unit comprising one or more memory units comprising instructions stored thereon and one or more processors, wherein the one or more processors are communicatively coupled to the one or more memory units, wherein the control unit is configured to receive or obtain the real-time neural signals from the recording device, wherein the one or more processors are programmed to execute the instructions to:
input the real-time neural signals into a post-trained machine learning model to generate control information based on brain-state embeddings,
wherein the post-trained machine learning model is obtained by:
pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model, and
post-training the pre-trained machine learning model using supervised learning on the sequences of neural signals or additional sequences of neural signals aligned with labeled contextual information to yield the post-trained machine learning model, wherein the labeled contextual information is obtained by recording contextual data from one or more modalities in real or simulated environments experienced by the one or more subjects or one or more users, wherein the labeled contextual information comprises descriptive representations derived from the one or more modalities obtained during at least one of:
passively observing an environment or activities of the one or more subjects or the one or more users, and
modifying the environment of the one or more subjects or the one or more users to induce targeted neural activity, wherein, during the post-training, the pre-trained machine learning model transforms the task-agnostic latent representations learned during the pre-training into the brain-state embeddings configured to align neural and contextual representations, and
control one or more devices based on the control information.

62.-80. (canceled)

81. A non-transitory computer-readable medium comprising instructions stored thereon that, when executed by one or more processors, cause the one or more processors to perform operations comprising:
inputting real-time neural signals recorded from a user into a post-trained machine learning model to generate control information based on brain-state embeddings,
wherein the post-trained machine learning model is obtained by:
pre-training a machine learning model using unsupervised or self-supervised learning on sequences of neural signals recorded from electrodes in or on one or more subjects, prior to associating the sequences of neural signals with task-specific labels, such that the machine learning model learns task-agnostic latent representations of neural activity to yield a pre-trained machine learning model, and
post-training the pre-trained machine learning model using supervised learning on the sequences of neural signals or additional sequences of neural signals aligned with labeled contextual information to yield the post-trained machine learning model, wherein the labeled contextual information is obtained by recording contextual data from one or more modalities in real or simulated environments experienced by the one or more subjects or one or more users, wherein the labeled contextual information comprises descriptive representations derived from the one or more modalities obtained during at least one of:
passively observing an environment or activities of the one or more subjects or the one or more users, and
modifying the environment of the one or more subjects or the one or more users to induce targeted neural activity, wherein, during the post-training, the pre-trained machine learning model transforms the task-agnostic latent representations learned during the pre-training into the brain-state embeddings configured to align neural and contextual representations; and
controlling one or more devices based on the control information.

82.-100. (canceled)
